(19) United States
(12) Patent Application Publication
(10) Pub. No.: US 2015/0370965 A1
(43) Pub. Date: Dec. 24, 2015

(54) MULTIPLE BIOMARKER PANELS TO STRATIFY DISEASE SEVERITY AND MONITOR TREATMENT OF DEPRESSION

(71) Applicant: Ridge Diagnostics, Inc., La Jolla, CA (US)

(72) Inventors: John Bilello, Durham, NC (US); Bo Pi, Carlsbad, CA (US)

(21) Appl. No.: 14/746,379

(22) Filed: Jun. 22, 2015

Related U.S. Application Data
(63) Continuation of application No. 14/299,303, filed on Jun. 9, 2014, now abandoned.
Application No. 14/299,303 is a continuation-in-part of application No. 13/312,553, filed on Dec. 6, 2011, now abandoned.
Application No. 14/299,303 is a continuation-in-part of application No. 13/014,413, filed on Jan. 26, 2011, now abandoned.
Application No. 14/299,303 is a continuation-in-part of application No. 12/754,770, filed on Apr. 6, 2010, now abandoned.
Application No. 14/299,303 is a continuation-in-part of application No. 12/753,022, filed on Apr. 1, 2010, now abandoned.
(60) Provisional application No. 61/420,141, filed on Dec. 6, 2010.
(60) Provisional application No. 61/298,443, filed on Jan. 26, 2010.
(60) Provisional application No. 61/166,986, filed on Apr. 6, 2009.
(60) Provisional application No. 61/165,662, filed on Apr. 1, 2009.

(51) Int. Cl. G06F 19/24; A61N 2/00; A61M 21/02; A61K 31/343

(52) U.S. Cl. CPC G06F 19/24; A61K 31/343; A61N 2/004; A61M 21/02

(57) ABSTRACT

Materials and methods for identifying and measuring pharmacodynamic biomarkers of neuropsychiatric disease (e.g., major depressive disorder), stratifying disease severity, and monitoring a subject's response to treatment are provided.

### CROSS-REFERENCE TO RELATED APPLICATIONS

This application is a continuation of U.S. application Ser. No. 14/299,303, filed on Jun. 9, 2014 which is:

(a) a continuation-in-part of U.S. application Ser. No. 13/312,553, filed on Dec. 6, 2011, which claims benefit of priority from U.S. Provisional Application No. 61/420,141, filed Dec. 6, 2010;

(b) a continuation-in-part of U.S. application Ser. No. 13/014,413, filed on Jan. 26, 2011, which claims benefit of priority from U.S. Provisional Application Ser. No. 61/298,443, filed on Jan. 26, 2010;

(c) a continuation-in-part of U.S. application Ser. No. 12/754,770, filed Apr. 6, 2010, which claims benefit of priority from U.S. Provisional Application Ser. No. 61/166,986, filed on Apr. 6, 2009; and

(d) a continuation-in-part of U.S. application Ser. No. 12/753,022, filed Apr. 1, 2010, which claims benefit of priority from U.S. Provisional Application Ser. No. 61/165,662, filed on Apr. 1, 2009.

Each of the above-referenced applications is incorporated herein by reference in its entirety.

### BACKGROUND

1. Technical Field

This document relates to materials and methods for stratifying disease severity and monitoring the effectiveness of treatment in a subject having neuropsychiatric disease (e.g., in a depressed individual).

2. Background Information

Neuropsychiatric diseases include major depression, schizophrenia, mania, post-traumatic stress disorder, Tourette's disorder, Parkinson's disease, and obsessive compulsive disorder. These disorders often are debilitating and difficult to diagnose and treat effectively. Neuropsychiatric conditions are the world's leader in “years lived with disability” (YLDs), accounting for almost 30% of total YLDs. Unipolar major depressive disorder (MDD) alone accounts for 11% of global YLDs.

Several factors, including inaccurate diagnosis, early discontinuation of treatment, and inadequate antidepressant dosing, may contribute to sustained disability and sub-optimal treatment outcomes. For example, nearly one-half of medical outpatients who receive an antidepressant prescription discontinue treatment during the first month. Discontinuation rates within the first three months of treatment can reach 68%, depending on the population studied and the therapeutic agent employed. Efforts to provide optimal treatment to patients with MDD and other neuropsychiatric conditions are often stymied by the traditional reliance upon clinical assessments and the patient's self-reporting of symptoms for diagnosis and for monitoring the efficacy of treatment. Such methods are subjective and often unreliable. Further, most clinical disorders do not arise due to a single biological change, but rather are the result of interactions between multiple factors. Different individuals affected by the same clinical condition (e.g., major depression) may present with a different range or extent of symptoms, depending on the specific changes within each individual.

### SUMMARY

This document is based in part on the development of methods for identifying pharmacodynamic biomarkers of neuropsychiatric disease that can be used for monitoring a subject's response to treatment.

For many neuropsychiatric diseases, the only means of diagnosis and monitoring of treatment is clinical evaluation. Traditional reliance upon clinical assessments and patient interviews for diagnosing neuropsychiatric diseases and establishing and monitoring treatment can be associated with sub-optimal outcomes for many patients. The ability to determine disease status on an individual basis would permit accurate assessment of a subject's individual disease state. There is a need for reliable methods for diagnosing clinical (e.g., neuropsychiatric) conditions, assessing disease status, and monitoring response to treatment. In addition, rational design and application of new therapeutics for neuropsychiatric diseases requires the discovery, validation, and implementation of informative indicators of biological processes or pharmacological responses to therapeutic intervention. It would be advantageous for clinicians and other mental health professionals to use quantitative diagnostic parameters based on physiological measurements to supplement or replace clinical assessments and patient interviews in order to diagnosis depression, accurately stratify disease severity, and monitor the patient's response to treatment.

This document is based in part on the identification of quantitative biomarkers that are indicative of disease (e.g., neuropsychiatric disease) and can be used to measure the impact of therapeutic intervention. This document also is based in part on the development of methods for diagnosing depressive disorders, stratifying disease severity, and monitoring response to treatment. Biomarkers as disclosed herein can be useful for clinicians and other mental health professionals in the diagnosis and assessment of neuropsychiatric disorders.

As described herein, this document also provides materials and methods for establishing a baseline diagnosis of depression by developing an algorithm, evaluating multiple parameters, and using the algorithm to determine a set of quantitative diagnostic scores. The approach described herein differs from some of the more traditional approaches to using biomarkers in the construction of an algorithm versus analyzing single markers or groups of markers. As provided herein, algorithms can be used to derive a single value that reflects a particular disease state, prognosis, or response to treatment. Highly multiplexed microarray-based immunological tools can be used to simultaneously measure multiple parameters. In this manner, all results can be derived simultaneously from the same sample and under the same conditions. High-level pattern recognition techniques can be applied using widely available tools such as hierarchical clustering, self-organizing maps, and supervised classification algorithms (e.g., support vector machines, k-nearest neighbors, and neural networks). The latter group of analytical approaches is likely to have substantial value for clinical use of the materials and methods provided herein.

In one aspect, this document features a method for identifying biomarkers of neuropsychiatric disease, comprising (a) calculating a first diagnostic disease score for a subject having said neuropsychiatric disease, wherein said first diagnostic disease score is calculated prior to administration of transcranial magnetic stimulation to said subject; (b) providing numerical values for the levels of one or more analytes in a first biological sample obtained from said subject prior to administration of said transcranial magnetic stimulation; (c) calculating a second diagnostic disease score for said subject after administration of said transcranial magnetic stimulation; (d) providing numerical values for the levels of said one or more analytes in a second biological sample obtained from said subject after administration of said transcranial magnetic stimulation; and (e) identifying one or more analytes as being biomarkers for said neuropsychiatric disease, wherein said one or more analytes are identified as biomarkers if they are differentially expressed between said first and second biological samples, wherein said differential expression of said one or more analytes correlates to a positive or negative change in said subject's diagnostic score.

The neuropsychiatric disease can be major depressive disorder (MDD). The diagnostic scores can be determined by clinical assessment. An analyte can be identified as being a biomarker for the neuropsychiatric disease if the expression level of the analyte is correlated with a positive or negative change in the second diagnostic score relative to the first diagnostic score. The administration of transcranial magnetic stimulation can comprise repetitive transcranial magnetic stimulation. The administration of transcranial magnetic stimulation can comprise stimulating a prefrontal cortex of the subject. The first and second biological samples can be selected from the group consisting of blood, serum, cerebrospinal fluid, plasma, and lymphocytes. The second biological sample can be collected from the subject hours, days, weeks, or months after administering transcranial magnetic stimulation to the subject. Steps (c), (d), and (e) can be repeated at intervals of time after administering transcranial magnetic stimulation to the subject. The subject also can be monitored using molecular imaging technology and/or clinical evaluation tools such as the Hamilton Rating Scale for Depression (HAM-D) Score. The subject can receive one or more additional forms of therapeutic intervention (e.g., one or more additional forms of therapeutic intervention selected from the group consisting of cognitive behavioral therapy, drug therapy, therapeutic interventions that are behavioral in nature, group therapies, interpersonal therapies, psychodynamic therapies, relaxation or meditative therapies, and traditional psychotherapy). The method can further comprise providing the first and second biological samples from the subject, and/or administering transcranial magnetic stimulation to the subject. The method can be a computer-implemented method. In some embodiments, the method can further comprise (f) using biomarker hypermapping technology to identify specific groups of analytes that are differentially expressed between the first and second biological samples, wherein the differential expression of a group of analytes correlates to a positive or negative change in the subject's hyperspace pattern.

In another aspect, this document features a method for identifying biomarkers of neuropsychiatric disease, comprising (a) providing a first biological sample from a subject; (b) determining the subject's first diagnostic disease score; (c) administering transcranial magnetic stimulation to the subject; (d) providing a second biological sample from the subject obtained following transcranial magnetic stimulation, and determining expression of one or more analytes in the first biological sample and the second biological sample; (e) determining the subject's second diagnostic disease score following the transcranial magnetic stimulation; and (f) identifying one or more analytes as being biomarkers for the neuropsychiatric disease, wherein the one or more analytes are identified as biomarkers if they are differentially expressed between the first and second biological samples, wherein the differential expression of the one or more analytes correlates to a positive or negative change in the subject's diagnostic score.

The neuropsychiatric disease can be MDD. The diagnostic scores can be determined by clinical assessment. The administration of transcranial magnetic stimulation can comprise repetitive transcranial magnetic stimulation. The administration of transcranial magnetic stimulation can comprise stimulating a prefrontal cortex of the subject. The first and second biological samples can be selected from the group consisting of blood, serum, cerebrospinal fluid, plasma, and lymphocytes. The second biological sample can be collected from the subject hours, days, weeks, or months after administering transcranial magnetic stimulation to the subject. Steps (c), (d), and (e) can be repeated at intervals of time after administering transcranial magnetic stimulation to the subject. The method can further comprise monitoring the subject using molecular imaging technology. The method can further comprise administering one or more additional forms of therapeutic intervention to the subject. The one or more additional forms of therapeutic intervention can be selected from the group consisting of cognitive behavioral therapy, drug therapy, therapeutic interventions that are behavioral in nature, group therapies, interpersonal therapies, psychodynamic therapies, relaxation or meditative therapies, and traditional psychotherapy. The method can be a computer-implemented method.

This document also features a method for assessing a treatment response in a mammal having a neuropsychiatric disease, comprising (a) determining a first diagnostic disease score for the mammal, wherein the first diagnostic disease score is calculated using numerical values for the levels of at least two inflammatory markers, at least two HPA axis markers, and at least two metabolic markers present in a first biological sample obtained from the mammal prior to administration of the treatment; (b) determining a second diagnostic disease score for the mammal, wherein the second diagnostic disease score is calculated using numerical values for the levels of at least two inflammatory markers, at least two HPA axis markers, and at least two metabolic markers present in a second biological sample obtained from the mammal after administration of the treatment; and (c) maintaining, adjusting, or stopping the treatment of the mammal based on a comparison of the first diagnostic disease score to the second diagnostic disease score. The mammal can be a human. The treatment can be transcranial magnetic stimulation. The first diagnostic disease score can be calculated using numerical values for the levels of at least two inflammatory markers, at least two HPA axis markers, at least two metabolic markers, and at least two neurotrophic markers present in the first biological sample. The second diagnostic disease score can be calculated using numerical values for the levels of at least two inflammatory markers, at least two HPA axis markers, at least two metabolic markers, and at least two neurotrophic markers present in the second biological sample. The method can include using a hypermap that comprises using a score for the levels of the inflammatory markers, a score for the levels of the at least two HPA axis markers, and a score for the levels of the at least two metabolic markers to compare the first and second diagnostic disease scores.

In still another aspect, this document features a method for identifying biomarkers of neuropsychiatric disease, comprising (a) calculating a first diagnostic disease score for a subject having said neuropsychiatric disease, wherein said first diagnostic disease score is calculated prior to administration of vagus nerve stimulation to said subject; (b) providing numerical values for the levels of one or more analytes in a first biological sample obtained from said subject prior to administration of said vagus nerve stimulation; (c) calculating a second diagnostic disease score for said subject after administration of said vagus nerve stimulation; (d) providing numerical values for the levels of said one or more analytes in a second biological sample obtained from said subject after administration of said vagus nerve stimulation; and (e) identifying one or more analytes as being biomarkers for said neuropsychiatric disease, wherein said one or more analytes are identified as biomarkers if they are differentially expressed between said first and second biological samples, wherein said differential expression of said one or more analytes correlates to a positive or negative change in said subject's diagnostic score.

The neuropsychiatric disease can be major depressive disorder (MDD). The diagnostic scores can be determined by clinical assessment. An analyte can be identified as being a biomarker for the neuropsychiatric disease if the expression level of the analyte is correlated with a positive or negative change in the second diagnostic score relative to the first diagnostic score. The administration of vagus nerve stimulation can comprise repetitive vagus nerve stimulation. The first and second biological samples can be selected from the group consisting of blood, serum, cerebrospinal fluid, plasma, and lymphocytes. The second biological sample can be collected from the subject hours, days, weeks, or months after administering vagus nerve stimulation to the subject. Steps (c), (d), and (e) can be repeated at intervals of time after administering vagus nerve stimulation to the subject. The subject can be monitored using molecular imaging technology and/or clinical evaluation tools such as the Hamilton Rating Scale for Depression (HAM-D) score. The subject can receive one or more additional forms of therapeutic intervention (e.g., one or more additional forms of therapeutic intervention selected from the group consisting of cognitive behavioral therapy, drug therapy, therapeutic interventions that are behavioral in nature, group therapies, interpersonal therapies, psychodynamic therapies, relaxation or meditative therapies, and traditional psychotherapy). The method can further comprise providing the first and second biological samples from the subject, and/or administering vagus nerve stimulation to the subject. The method can be a computer-implemented method.

In another aspect, this document features a method for identifying biomarkers of neuropsychiatric disease, comprising (a) providing a first biological sample from a subject; (b) determining the subject's first diagnostic disease score; (c) administering vagus nerve stimulation to the subject; (d) providing a second biological sample from the subject obtained following vagus nerve stimulation, and determining expression of one or more analytes in the first biological sample and the second biological sample; (e) determining the subject's second diagnostic disease score following the vagus nerve stimulation; and (f) identifying one or more analytes as being biomarkers for the neuropsychiatric disease, wherein the one or more analytes are identified as biomarkers if they are differentially expressed between the first and second biological samples, wherein the differential expression of the one or more analytes correlates to a positive or negative change in the subject's diagnostic score.

The neuropsychiatric disease can be MDD. The diagnostic scores can be determined by clinical assessment. The administration of vagus nerve stimulation can comprise repetitive vagus nerve stimulation. The first and second biological samples can be selected from the group consisting of blood, serum, cerebrospinal fluid, plasma, and lymphocytes. The second biological sample can be collected from the subject hours, days, weeks, or months after administering vagus nerve stimulation to the subject. Steps (c), (d), and (e) can be repeated at intervals of time after administering vagus nerve stimulation to the subject. The method can further comprise monitoring the subject using molecular imaging technology. The method can further comprise administering one or more additional forms of therapeutic intervention to the subject. The one or more additional forms of therapeutic intervention can be selected from the group consisting of cognitive behavioral therapy, drug therapy, therapeutic interventions that are behavioral in nature, group therapies, interpersonal therapies, psychodynamic therapies, relaxation or meditative therapies, and traditional psychotherapy. The method can be a computer-implemented method.

This document also features a method for assessing a treatment response in a mammal having a neuropsychiatric disease, comprising (a) determining a first diagnostic disease score for the mammal, wherein the first diagnostic disease score is calculated using numerical values for the levels of at least two inflammatory markers, at least two HPA axis markers, and at least two metabolic markers present in a first biological sample obtained from the mammal prior to administration of the treatment; (b) determining a second diagnostic disease score for the mammal, wherein the second diagnostic disease score is calculated using numerical values for the levels of at least two inflammatory markers, at least two HPA axis markers, and at least two metabolic markers present in a second biological sample obtained from the mammal after administration of the treatment; and (c) maintaining, adjusting, or stopping the treatment of the mammal based on a comparison of the first diagnostic disease score to the second diagnostic disease score. The mammal can be a human. The treatment can be vagus nerve stimulation. The first diagnostic disease score can be calculated using numerical values for the levels of at least two inflammatory markers, at least two HPA axis markers, at least two metabolic markers, and at least two neurotrophic markers present in the first biological sample. The second diagnostic disease score can be calculated using numerical values for the levels of at least two inflammatory markers, at least two HPA axis markers, at least two metabolic markers, and at least two neurotrophic markers present in the second biological sample. The method can include using a hypermap that comprises using a score for the levels of the inflammatory markers, a score for the levels of the at least two HPA axis markers, and a score for the levels of the at least two metabolic markers to compare the first and second diagnostic disease scores.

In another aspect, this document features a method for stratifying disease severity in a subject, comprising: (a) providing a numerical value for each of a plurality of analytes relevant to mild, moderate, and severe states of depression, wherein each numerical value corresponds to the level of the analyte in a biological sample from the subject; (b) individually weighting each numerical value in a manner specific to each analyte to obtain a weighted value for each analyte; (c) determining a result value based on an equation that includes each weighted value; (d) comparing the result value to control result values obtained for a normal subject and for subjects having mild, moderate, and severe states of depression, wherein the control result values were determined in a manner comparable to that of the result value; and (e) if the result value is within a predetermined range of control values for no depression, mild depression, moderate depression, or severe depression, classifying the subject having no depression, mild depression, moderate depression, or severe depression, respectively.

The depression can be associated with MDD. An algorithm can be used to calculate a MDD diagnostic score that can be used to support the classification of mild, moderate, and severe states of MDD. The plurality of analytes can include one or more inflammatory biomarkers, one or more neurotrophic biomarkers, one or more metabolic biomarkers, and/or one or more hypothalamic-pituitary-adrenal axis biomarkers. The plurality of analytes can include two or more analytes selected from the group consisting of acylation stimulating protein, adiponectin, adrenocorticotropic hormone, artemin, alpha 1 antitrypsin (A1AT), alpha-2-macroglobin, apolipoprotein C3 (ApoC3), arginine vasopressin, brain-derived neurotrophic factor (BDNF), corticotropin-releasing hormone, C-reactive protein, CD40 ligand, cortisol, epidermal growth factor (EGF), granulocyte colony-stimulating factor, interleukin-1, interleukin-1 receptor agonist, interleukin-6, interleukin-10, interleukin-13, interleukin-18, leptin, macrophage inflammatory protein 1-alpha, myeloperoxidase (MPO), neurotrophin 3, pancreatic polypeptide, plasminogen activator inhibitor-1, prolactin, RANTES, resistin, reelin, S100B, soluble tumor necrosis factor alpha, soluble tumor necrosis factor alpha receptor II (sTNFR2), thyroid stimulating hormone, tumor necrosis factor alpha, or a combination thereof. For example, the plurality of analytes can include cortisol, prolactin, EGF, MPO, BDNF, resistin, sTNFR2, ApoC3, and A1AT. The biological sample can be whole blood, serum, plasma, urine, or cerebrospinal fluid. The subject can be a human. In addition, the method can further include obtaining a measured level of one or more of the plurality of analytes for the biological sample, and the result value can be based at least in part on the measured level.

In another aspect, this document features a method for monitoring treatment of a subject diagnosed with a depressive disorder, comprising (a) providing a first numerical value of each of a plurality of analytes relevant to depression, wherein each first numerical value corresponds to the level of the analyte in a first biological sample from the subject; (b) individually weighting each first numerical value in a manner specific to each analyte to obtain a first weighted value for each analyte; (c) determining a first MDD score based on an equation that includes each first weighted value; (d) providing a second numerical value of each of the plurality of analytes, wherein each second numerical value corresponds to the level of the analyte in a second biological sample from the subject, wherein the second biological sample is obtained after treatment for the depressive disorder; (e) individually weighting each second numerical value in a manner specific to each analyte to obtain a second weighted value for each analyte, with the proviso that the weighting is done in a manner comparable to that in step (b); (f) using the equation to determine a second MDD score after treatment of the subject for the depressive disorder; and (g) comparing the first MDD score to the second MDD score and to a control MDD score or range of MDD scores determined from one or more normal subjects, and classifying the treatment as being effective if the second MDD score is closer than the first MDD score to the control MDD score, or classifying the treatment as not being effective if the second MDD score is not closer than the first MDD score to the control MDD score.

The biological sample can be whole blood, serum, plasma, urine, or cerebrospinal fluid. The second MDD score can be determined days, weeks, or months after treatment for depression. The plurality of analytes can be selected from the group consisting of (a) RANTES, PRL, BDNF, S100B, RES, TNFR, A1A, cortisol, and EGF; (b) RANTES, PRL, BDNF, S100B, RES, TNFR, A1A, and EGF; (c) RANTES, PRL, BDNF, S100B, RES, TNFR, and A1A; (d) S100B, PRL, BDNF, RES, TNFR, and A1A; (e) cortisol, PRL, BDNF, RES, TNFR, and A1A; and (f) BDNF, resistin, TNFRII, and A1A. The subject can be a human. In some case, the method can further include obtaining a measured level of one or more of the plurality of analytes for the first or second biological sample, and the corresponding first or second MDD score can be based at least in part on the measured level.

In another aspect, this document features a method for monitoring treatment of a subject diagnosed with a depressive disorder, comprising (a) providing a first numerical value of each of a plurality of analytes relevant to depression, wherein each first numerical value corresponds to the level of the analyte in a first biological sample from the subject; (b) providing a second numerical value of each of the plurality of analytes, wherein each second numerical value corresponds to the level of the analyte in a second biological sample from the subject, wherein the second biological sample is obtained after treatment for the depressive disorder; (c) individually weighting the first and second numerical values in a manner specific to each analyte to obtain a weighted value for each analyte; (d) determining a monitoring score based on an equation that includes the weighted numerical values; and (e) comparing the monitoring score to a control monitoring score, and classifying the treatment as being effective if the monitoring score is greater than or equal to the control monitoring score, or classifying the treatment as not being effective if the monitoring score is less than the control monitoring score.

The biological sample can be whole blood, serum, plasma, urine, or cerebrospinal fluid. The first biological sample can be obtained from the subject before the start of the treatment, and the second biological sample can be obtained from the subject one to 25 days after start of the treatment. The method can further include providing a third numerical value of each of the plurality of analytes, wherein each third numerical value corresponds to the level of the analyte in a third biological sample from the subject; individually weighting the third numerical values in a manner specific to each analyte to obtain a weighted value for each analyte; and determining the monitoring score based on an equation that includes the first, second, and third weighted numerical values for each analyte. The plurality of analytes can be selected from the group consisting of (a) PRL, BDNF, RES, TNFRII, and A1A; and (b) RANTES, PRL, BDNF, S100B, RES, TNFR, A1A, and EGF.

Unless otherwise defined, all technical and scientific terms used herein have the same meaning as commonly understood by one of ordinary skill in the art to which this disclosure pertains. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a flow diagram showing steps that can be taken to identify disease-related biomarkers using defined patient populations and a biomarker library with or without the addition of disease-related content.

FIG. 2 is a flow diagram showing steps that can be taken to identify pharmacodynamic biomarkers that indicate a positive or negative response to treatment for a neuropsychiatric disease.

FIG. 3 is a flow diagram showing steps that can be taken to establish a set of pharmacodynamic biomarkers using mass spectroscopy-based differential protein measurement.

FIG. 4 is a graph plotting HAM-D scores and MDD scores (MDDSCORE™) derived from an algorithm applied to serum protein measurement prior to and after therapy. MDD patients prior to initiation of therapy are indicated by filled circles. The same MDD patient treated for 2 weeks with LEXAPRO™ are indicated by open squares, and the arrows indicate the direction of the shift in HAM-D Score and MDDSCORE™. Normal subjects at baseline are indicated as open circles.

FIG. 5 is a biomarker hypermap (BHYPERMAP™) of a dataset used to derive the MDDSCORE™ in a study of 50 MDD patients (filled circles) and 20 normal subjects (open circles).

FIG. 6 is a biomarker hypermap (BHYPERMAP™) of changes in patient map positions indicative of a positive or negative response to treatment for a neuropsychiatric disease. Treatment (Rx) was with LEXAPRO™. MDD patients at baseline are indicated by filled circles. Filled triangles represent patients after 2-3 weeks of treatment, and open squares represent patients after 8 weeks of treatment. The open circles represent untreated normal subjects.

FIG. 7 shows an example of a computer-based diagnostic system employing the biomarker analysis described in this document.

FIG. 8 shows an example of a computer system that can be used in the computer-based diagnostic system depicted in FIG. 7.

FIG. 9 is a flow diagram showing steps that can be taken to develop a diagnostic or prognostic algorithm using a disease-specific biomarker panel.

FIG. 10 is a flow diagram showing steps in an exemplary method for determining a basic diagnostic score. FIG. 11 is a flow diagram showing exemplary steps for using a diagnostic score to diagnose an individual, to select treatment options, and to monitor and optimize treatment.

FIG. 12 is a hypothetical box whisker plot of marker X levels in the blood of patients prior to and following anti-depressive therapy.

FIG. 13 is a graph plotting the correlation between depression diagnostic scores (MDDSCORE™) and Hamilton Depression Rating Scale (HDRS or HAM-D) scores for a group of normal subjects (filled circles) and a group of MDD patients (open circles).

FIG. 14 is a graph plotting patient HAM-D scores at both 2 and 8 weeks after treatment with the antidepressant Lexapro. A decrease in HAM-D score indicates improvement.

FIG. 15 is a graph plotting the change in depression diagnostic score (MDDSCORE™) in a subset of MDD patients at baseline and after 2 weeks of treatment with Lexapro.

FIG. 16 is a graph plotting the potential for the methods disclosed herein to predict efficacy of treatment at 8 weeks by determining the MDDSCORE™ after 2 weeks of treatment.

FIG. 17 is a flow diagram showing exemplary steps for using an algorithm to monitor treatment outcome in MDD patients.

FIG. 18 is a graph plotting the outcome of a treatment prediction prototype in which biomarker measurements obtained during the first two weeks of treatment were used to calculate a monitoring score to predict the outcome after eight weeks of treatment.

FIG. 19 is a graph plotting Hamilton Depression (HAM-D) Rating Scale scores (left panel) and Montgomery-Asberg Depression Rating Scale (MADRS) scores (right panel) for Korean drug-free MDD patients prior to and during treatment with LEXAPRO™ for a period of 8 weeks.

FIG. 20A-20E are graphs plotting levels of individual biomarkers in Korean MDD patients pre- and post-treatment with LEXAPRO™. FIG. 20A, brain-derived neurotrophic factor BDNF); FIG. 20B, cortisol; FIG. 20C, prolactin; FIG. 20D, resistin; FIG. 20E, soluble tumor necrosis factor alpha receptor II (sTNFαRII). Box plots of the individual biomarkers were obtained by direct measurement of the levels at baseline and at week two or three by quantitative immunoassay. The line across the box is the median value.

FIG. 21 is a graph plotting treatment outcome prediction using biomarker expression two weeks after treatment.

### DETAILED DESCRIPTION

This document is based in part on the identification of methods for establishing a diagnosis or prognosis of depression disorder conditions by developing an algorithm, evaluating (e.g., measuring) multiple parameters, and using the algorithm to determine a set of quantitative diagnostic scores. Algorithms for application of multiple biomarkers from biological samples such as serum or plasma can be developed for stratification of disease severity and identification of disease-specific pharmacodynamic markers. In some embodiments, algorithms for application of multiple biomarkers from biological samples such as, for example, cells, serum, or plasma can be developed for patient stratification, identification of pharmacodynamic markers, and monitoring treatment outcome.

As used herein, a “biomarker” is a characteristic that can be objectively measured and evaluated as an indicator of a normal biologic or pathogenic process or pharmacological response to a therapeutic intervention. Biomarker panels and their associated algorithms can encompass one or more analytes (e.g., proteins, nucleic acids, and metabolites), physical measurements, or combinations thereof. As used herein, an “analyte” is a substance or chemical constituent that can be objectively measured and determined in an analytical procedure such as immunoassay or mass spectrometry.

This document also is based in part on the identification of methods for diagnosing depressive disorder conditions and monitoring treatment by evaluating (e.g., measuring) biomarker expression. Thus, this document also provides methods for identifying treatment-responsive biomarkers. As described herein, this document provides methods and materials for identifying and validating pharmacodynamic biomarkers associated with positive or negative changes in a subject following treatment (e.g., by administration of transcranial magnetic stimulation (TMS) or vagus nerve stimulation (VNS)). As used herein, a “pharmacodynamic” biomarker is a biomarker that can be used to quantitatively evaluate (e.g., measure) the impact of treatment or therapeutic intervention on the course, severity, status, symptomology, or resolution of a disease. In some embodiments, pharmacodynamic biomarkers can be identified based on a correlation or the defined relationship between analyte expression levels and positive or negative changes in a subject's diagnostic score (e.g., HAM-D score in depression) relative to one or more pre-treatment baseline scores. In some cases, analyte expression levels can be measured in samples collected from a subject prior to and following treatment (e.g., with TMS or VNS, or mock stimulation). Analyte expression levels in pre-treatment (e.g., pre-TMS or pre-VNS) samples can be compared to analyte levels in post-treatment (e.g., post-TMS or post-VNS) samples. If the change in expression corresponds to positive or negative clinical outcomes, as determined by an improvement in the post-treatment diagnostic score relative to the pre-treatment diagnostic score, the analyte can be identified as pharmacodynamic biomarker for MDD and other neuropsychiatric diseases.

The methods and materials provided herein can be used to diagnose patients with neuropsychiatric disorders, determine treatment options, and provide quantitative measurements of treatment efficacy.

### Algorithms and Diagnostic Scores

This document provides methods and materials for determining a subject's diagnostic score. An exemplary subject for the methods described herein is a human, but subjects also can include animals that are used as models of human disease (e.g., mice, rats, rabbits, dogs, and non-human primates). The methods provided herein can be used to establish a baseline score prior to starting a new therapy regimen or continuing an existing therapy regimen. Diagnostic scores determined post-treatment can be compared to the baseline score in order to observe a positive or negative change relative to baseline.

Baseline and post-treatment diagnostic scores can be determined by any suitable method of assessment. For example, in MDD a clinical assessment of the subject's symptoms and well-being can be performed. The “gold standard” diagnostic method is the structured clinical interview. In some cases, a subject's diagnostic score can be determined using the clinically-administered Hamilton Depression Rating Scale (HAM-D), a 17-item scale that evaluates depressed mood, vegetative and cognitive symptoms of depression, and co-morbid anxiety symptoms. HAM-D can be used to quantify the severity of depressive symptoms at the time of assessment. See Michael Taylor & Max Fink, Melancholia: The Diagnosis, Pathophysiology, and Treatment of Depressive Illness, 91-92, Cambridge University Press (2006). Studies have demonstrated improved HAM-D scores following TMS and VNS. Other methods of clinical assessment can be used. In some cases, self-rating scales, such as the Beck Depression Inventory scale, can be used. Many rating scales for neuropsychiatric diseases are observer-based. For example, the Montgomery-Asberg Depression Rating Scale can be used to determine a subject's depression diagnostic score. To determine a diagnostic score based on a subject's overall social, occupational, and psychological functioning, the Global Assessment of Functioning Scale can be used.

In some cases, mathematical algorithms can be used to determine diagnostic scores. This document provides materials and methods for developing algorithms that incorporate multiple measured parameters and that can be used to determine a quantitative diagnostic score. Algorithms for determining an individual's disease status or response to treatment, for example, can be determined for any clinical condition. Algorithms for diagnosing or assessing response to treatment, for example, can be determined using metrics (e.g., serum levels of multiple analytes) associated with a defined clinical condition before and/or after treatment. As used herein, an “analyte” is a substance or chemical constituent that can be objectively measured and determined in an analytical procedure such as, without limitation, immunoassay or mass spectrometry. The algorithms provided herein can be mathematic functions containing multiple parameters that can be quantified using, for example, medical devices, clinical assessment scores, or biological, chemical, or physiological or physical tests of biological samples. Each mathematic function can be a weight-adjusted expression of the levels of parameters determined to be relevant to a selected clinical condition. Univariate and multivariate analyses can be performed on data collected for each marker using conventional statistical tools (e.g., not limited to: T-tests, PCA, LDA, or binary logistic regression). An algorithm can be applied to generate a set of diagnostic scores.

Algorithms generally can be expressed in the format of Formula 1:

Diagnostic score=f(x1, x2, x3, x4, x5 . . . xn) (1)

The diagnostic score is a value that is the diagnostic or prognostic result, “f” is any mathematical function, “n: is any integer (e.g., an integer from 1 to 10,000), and x1, x2, x3, x4, x5 . . . xn are the “n” parameters that are, for example, measurements determined by medical devices, clinical assessment scores, and/or test results for biological samples (e.g., human biological samples such as blood, serum, plasma, urine, or cerebrospinal fluid).

The parameters of an algorithm can be individually weighted. An example of such an algorithm is expressed in Formula 2:

Diagnostic score=a1*x1+a2*x2-a3*x3+a4*x4-a5*x5 (2)

Here, x1, x2, x3, x4, and x5 can be measurements determined by medical devices, clinical assessment scores, and/or test results for biological samples (e.g., human biological samples), and a1, a2, a3, a4, and a5 are weight-adjusted factors for x1, x2, x3, x4, and x5, respectively.

A diagnostic score can be used to quantitatively define a medical condition or disease, or the effect of a medical treatment. For example, an algorithm (e.g., an algorithm populated by a computer) can be used to determine a diagnostic score for a disorder such as depression. In such an embodiment, the degree of depression can be defined based on Formula 1, with the following general formula:

Depression diagnosis score=f(x1, x2, x3, x4, x5 . . . xn)

The depression diagnosis score is a quantitative number that can be used to measure the status or severity of depression in an individual, “f” is any mathematical function, “n” can be any integer (e.g., an integer from 1 to 10,000), and x1, x2, x3, x4, x5 . . . xn are, for example, the “n” parameters that are measurements determined using medical devices, clinical evaluation scores, and/or test results for biological samples (e.g., human biological samples).

In a more general form, multiple diagnostic scores Sm can be generated by applying multiple formulas to specific groupings of biomarker measurements, as illustrated in Formula 3:

Diagnostic scores Sm=Fm(x1, . . . xn) (3)

Multiple scores can be useful, for example, in the identification of specific types and subtypes of depressive disorders and/or associated disorders. In some cases, the depressive disorder is MDD. Multiple scores can also be parameters indicating patient treatment progress or the efficacy of the treatment selected. Diagnostic scores for subtypes of depressive disorders may help aid in the selection or optimization of antidepressants and other pharmaceuticals.

Biomarker expression level changes can be expressed in the format of Formula 4:

Cₘᵢ=M_{ib}−Mᵢₐ (4)

where M_{ib} and Mᵢₐ are expression levels of a biomarker before and after treatment, respectively. Change in a subject's diagnostic score can be expressed in the format of Formula 5:

H=HAM-D_{b}−HAM-Dₐ (5)

where HAM-D_{b} and HAM-Dₐ are diagnostic scores before and after treatment, respectively. A pre-established process can be used to select only subjects having a HAM-Dₐ score greater than a minimum cut-off value (Eh=efficacy cut-off value). Upon statistical evaluation, where statistical significance is defined as p<0.05, a biomarker having a p value less than 0.05 can be selected as a biomarker associated with therapy-responsive MDD.

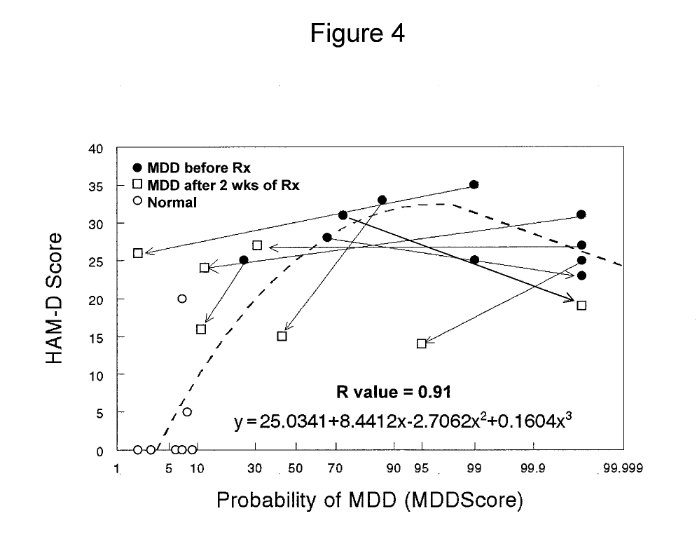

An example of how MDD scores and HAM-D scores can be used to monitor treatment-induced changes is shown in FIG. 4. The arrows indicate the directionality of change in scores from prior to treatment of MDD patients (filled circles) to after two weeks of treatment with LEXAPRO™ (open squares).

### Building Biomarker Libraries

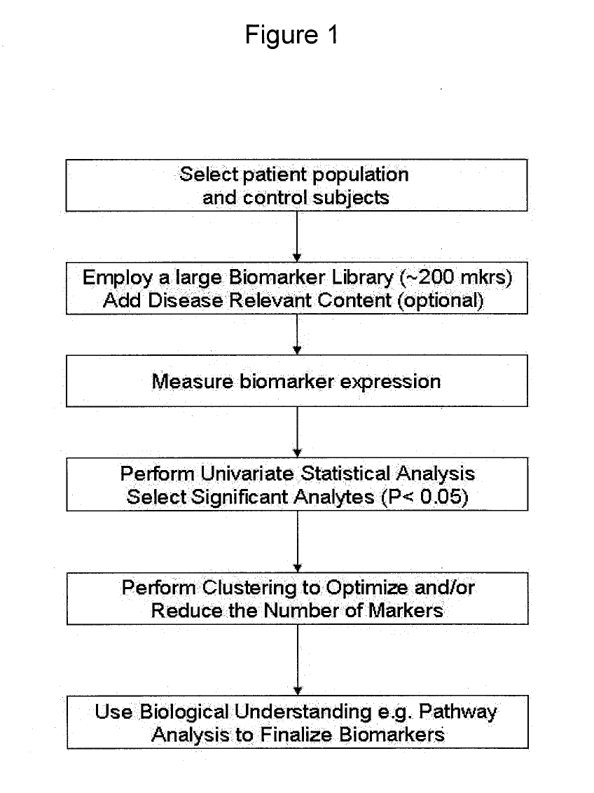

To determine what parameters are useful for inclusion in a diagnostic algorithm, a biomarker library of analytes can be developed, and individual analytes from the library can be evaluated for inclusion in an algorithm for a particular clinical condition. Pharmacodynamic biomarkers identified by the methods and materials provided herein can be previously unknown factors or biomolecules known to be associated with neuropsychiatric diseases. A procedure for using a biomarker library to identify potential neuropsychiatric biomarkers is diagrammed in FIG. 1. In some embodiments, as a starting point, a library can include analytes generally indicative of inflammation, cellular adhesion, immune responses, or tissue remodeling. In some embodiments (e.g., during initial library development), a library can include a dozen or more markers, a hundred markers, or several hundred markers. For example, a biomarker library can include a few hundred (e.g., about 200, about 250, about 300, about 350, about 400, about 450, or about 500) protein analytes. As a biomarker library is built, new markers can be added (e.g., markers specific to individual disease states, markers that are specific to the action of a particular therapeutic, and/or markers that are more generalized, such as growth factors). In some cases, a biomarker library can be refined by addition of disease related proteins obtained from discovery research (e.g., using differential display techniques, such as isotope coded affinity tags (ICAT) or mass spectroscopy). In this manner, a library can become increasingly specific to a particular disease state.

The addition of a new protein analyte to a biomarker library can require a purified or recombinant molecule, as well as an appropriate antibody (or antibodies) to capture and detect the new analyte. Addition of a new nucleic acid-based analyte to a biomarker library can require identification of a specific mRNA, as well as probes and detection systems to quantify the expression of that specific RNA. Although discovery of individual “new or novel” biomarkers is not necessary for developing useful algorithms, such markers can be included. Platform technologies that are suitable for multiple analyte detection methods as described herein typically are flexible and open to addition of new analytes. For example, the Luminex multiplex assay system (xMAP; luminexcorp.com on the World Wide Web) is flexible and can be readily configured to incorporate new disease-specific analytes.

While this document indicates that multiplexed detection systems can provide robust and reliable measurement of analytes relevant to diagnosing, stratifying, and monitoring clinical conditions, this does not preclude the use of assays capable of measuring the concentration of individual analytes from the panel (e.g., a series of single analyte ELISAs). Biomarker panels can be expanded and transferred to traditional protein arrays, multiplexed bead platforms or label-free arrays, and algorithms can be developed to support clinicians and clinical research.

Custom antibody array(s) can be designed, developed, and analytically validated for about 25-50 antigens. Initially, a panel of about 5 to 10 (e.g., 5, 6, 7, 8, 9, or 10) analytes can be chosen based on their ability to, for example, distinguish affected from unaffected subjects, or to stratify patients from a defined sample set according to disease severity. An enriched database, however, usually one in which more than 10 significant analytes are measured, can increase the sensitivity and specificity of test algorithms. Other panels can be run in addition to the panel reflecting inflammation and immune response to further define the disease state or sub-classify patients. By way of example, data obtained from measurements of neurotrophic factors can discern patients with alterations in neuroplasticity. Similarly, data obtained from measurements of metabolic factors can discern patients with altered or abnormal metabolic function, and data obtained from measurements of factors of the hypothalamic-pituitary-adrenal (HPA or HTPA) axis can discern patients with alterations of the neuroendocrine system. It is noted that such approaches also can include or be applied to other biological molecules including, without limitation, DNA and RNA.

### Selecting Individual Biomarkers

Many biomolecules are either up-regulated or down-regulated in subjects having different neuropsychiatric diseases. To determine what biomarkers are associated with different neuropsychiatric diseases, a biomarker library of analytes can be developed. In the construction of libraries or panels, markers and parameters can be selected by any of a variety of methods. The primary consideration for constructing a disease specific library or panel can be knowledge of a parameter's relevance to the disease. Literature searches or experimentation also can be used to identify other parameters/markers for inclusion. Numerous transcription factors, growth factors, hormones, and other biological molecules are associated with neuropsychiatric disorders. The parameters used to choose analytes or define biomarkers for MDD can be selected from, for example, the functional groupings of inflammatory biomarkers, HPA axis factors, metabolic biomarkers, and neurotrophic factors, including neurotrophins, glial cell-line derived neurotrophic factor family ligands (GFLs), and neuropoietic cytokines

In some cases, biomarkers can be factors involved in the inflammatory response. A wide variety of proteins are involved in inflammation, and any one of them is open to a genetic mutation that impairs or otherwise disrupts the normal expression and function of that protein. Inflammation also induces high systemic levels of acute-phase proteins. These proteins include C-reactive protein, serum amyloid A, serum amyloid P, vasopressin, and glucocorticoids, which cause a range of systemic effects. Inflammation also involves release of pro-inflammatory cytokines and chemokines Studies have demonstrated that abnormal functioning of the inflammatory response system disrupts feedback regulation of the immune system, thereby contributing to the development of neuropsychiatric and immunologic disorders. In fact, several medical illnesses that are characterized by chronic inflammatory responses (e.g., rheumatoid arthritis) have been reported to be accompanied by depression. Furthermore, recent evidence has linked elevated levels of inflammatory cytokines with both depression and cachexia, and experiments have shown that introducing cytokines induces depression and cachectic symptoms in both humans and rodents, suggesting that there may be a common etiology at the molecular level. For example, administration of pro-inflammatory cytokines (e.g., in cancer or hepatitis C therapies) can induce “sickness behavior” in animals, which is a pattern of behavioral alterations that is very similar to the behavioral symptoms of depression in humans. Therapeutic agents targeting specific cytokine molecules, such as tumor necrosis factor-alpha, are currently being evaluated for their potential to simultaneously treat both depression and cachexia pharmacologically. In sum, the “Inflammatory Response System (IRS) model of depression” (Maes, Adv. Exp. Med. Biol. 461:25-46 (1999)) proposes that pro-inflammatory cytokines, acting as neuromodulators, represent key factors in mediation of the behavioral, neuroendocrine and neurochemical features of depressive disorders.

Table 1 provides an exemplary, non-limiting list of inflammatory biomarkers.

TABLE 1

| Gene Symbol | Gene Name | Cluster |
|---|---|---|
| A1AT | Alpha 1 Antitrypsin | Inflammation |
| A2M | Alpha 2 Macroglobulin | Inflammation |
| AGP | Alpha 1-Acid Glycoprotein | Inflammation |
| ApoC3 | Apolipoprotein CIII | Inflammation |
| CD40L | CD40 ligand | Inflammation |
| IL-1(α or β) | Interleukin 1 | Inflammation |
| IL-6 | Interleukin 6 | Inflammation |
| IL-13 | Interleukin 13 | Inflammation |
| IL-18 | Interleukin 18 | Inflammation |
| IL-1ra | Interleukin 1 Receptor Antagonist | Inflammation |
| MPO | Myeloperoxidase | Inflammation |
| PAI-1 | Plasminogen activator inhibitor-1 | Inflammation |
| RANTES | RANTES (CCL5) | Inflammation |
| TNFA | Tumor Necrosis Factor alpha | Inflammation |
| STNFR | Soluble TNFα receptor (I, II) | Inflammation |

In some cases, biomarkers can be neurotrophic factors. Neurotrophic factors are a family of proteins that are responsible for the growth and survival of developing neurons and the maintenance of mature neurons. Most neurotrophic factors belong to one of three families: (1) neurotrophins, (2) glial cell-line derived neurotrophic factor family ligands (GFLs), and (3) neuropoietic cytokines Each family has its own distinct signaling family, yet the cellular responses elicited often overlap. Neurotrophic factors such as brain-derived neurotrophic factor (BDNF) and its receptor, TrkB, are proteins responsible for the growth and survival of developing neurons and for the maintenance of mature neurons. Neurotrophic factors can promote the initial growth and development of neurons in the CNS and PNS, as well as regrowth of damaged neurons in vitro and in vivo. In addition, these factors often are released by a target tissue in order to guide the growth of developing axons. Studies have suggested that deficits in neurotrophic factor synthesis may be responsible for increased apoptosis in the hippocampus and prefrontal cortex that is associated with the cognitive impairment described in depression.

Table 2 provides an exemplary, non-limiting list of neurotrophic biomarkers.

TABLE 2

| Gene Symbol | Gene Name | Cluster |
|---|---|---|
| BDNF | Brain-derived neurotrophic factor | Neurotrophic |
| S100B | S100B | Neurotrophic |
| NTF3 | Neurotrophin 3 | Neurotrophic |
| RELN | Reelin | Neurotrophic |
| GDNF | Glial cell line derived neurotrophic factor | Neurotrophic |
| ARTN | Artemin | Neurotrophic |

In some cases, biomarkers can be factors of the HPA axis. The HPA axis, also known as the limbic-hypothalamic-pituitary-adrenal axis (LHPA axis), is a complex set of direct influences and feedback interactions among the hypothalamus (a hollow, funnel-shaped part of the brain), the pituitary gland (a pea-shaped structure located below the hypothalamus), and the adrenal (or suprarenal) glands (small, conical organs on top of the kidneys). Interactions among these organs constitute the HPA axis, a major part of the neuroendocrine system that controls the body's stress response and regulates many body processes, including digestion, the immune system, mood and emotions, sexuality, and energy storage and expenditure. Examples of HPA axis biomarkers include ACTH and cortisol. Cortisol inhibits secretion of corticotropin-releasing hormone (CRH), resulting in feedback inhibition of ACTH secretion. This normal feedback loop may break down when humans are exposed to chronic stress, and may be an underlying cause of depression.

Table 3 provides an exemplary, non-limiting list of HPA axis biomarkers.

TABLE 3

| Gene Symbol | Gene Name | Cluster |
|---|---|---|
| None | Cortisol | HPA axis |
| EGF | Epidermal Growth Factor | HPA axis |
| GCSF | Granulocyte Colony Stimulating Factor | HPA axis |
| PPY | Pancreatic Polypeptide | HPA axis |
| ACTH | Adrenocorticotropic hormone | HPA axis |
| AVP | Arginine Vasopressin | HPA axis |
| CRH | Corticotropin-Releasing Hormone | HPA axis |

In some cases, biomarkers can be metabolic factors. Metabolic biomarkers are a group of biomarkers that provide insight into metabolic processes in wellness and disease states. Human diseases manifest in complex downstream effects, affecting multiple biochemical pathways. For example, depression and other neuropsychiatric disorders often are associated with metabolic disorders such as diabetes. Consequently, various metabolites and the proteins and hormones controlling metabolic processes can be used for diagnosing depressive disorders such as MDD, stratifying disease severity, and monitoring a subject's response to treatment for the depressive disorder.

Table 4 provides an exemplary, non-limiting list of metabolic biomarkers.

TABLE 4

| Gene Symbol | Gene Name | Cluster |
|---|---|---|
| ACRP30 | Adiponectin | Metabolic |
| ASP | Acylation Stimulating Protein | Metabolic |
| FABP | Fatty Acid Binding Protein | Metabolic |
| INS | Insulin | Metabolic |
| LEP | Leptin | Metabolic |
| PRL | Prolactin | Metabolic |
| RETN | Resistin | Metabolic |
| None | Testosterone | Metabolic |
| TSH | Thyroid Stimulating Hormone | Metabolic |
| None | Thyroxine | Metabolic |

In some cases, biomarkers for MDD can be selected from a panel of analytes that includes alpha-2-macroglobulin (A2M), acylation stimulating protein (ASP), BDNF, C-reactive protein (CRP), cortisol, epidermal growth factor (EGF), interleukin 1 (IL-1) interleukin-6 (IL-6), interleukin-10 (IL-10), interleukin-18 (IL-18), leptin, macrophage inflammatory protein 1-alpha (MIP-1α), myeloperoxidase (MPO), neurotrophin 3 (NT-3), plasminogen activator inhibitor-1 (PAI-1), prolactin (PRL), RANTES, resistin (RES), S100B protein, soluble TNFα receptor II) (sTNFαRII), tumor necrosis factor alpha (TNF-α), alpha 1 antitrypsin (A1AT), apolipoprotein CIII (ApoCIII), and any combination thereof. For example, a biomarker panel can include any two or more (e.g., two, three, four, five, six, seven, eight, nine, ten, or more) of the analytes disclosed herein.

In some cases, biomarkers for MDD can be a panel of analytes including one or more of acylation stimulating protein (ASP), adiponectin (ACRP30), adrenocorticotropic hormone (ACTH), artemin (ARTN), alpha 1 antitrypsin (A1AT), alpha-2-macroglobin (A2M), apolipoprotein C3 (apoC3), arginine vasopressin (AVP), brain-derived neurotrophic factor (BDNF), corticotropin-releasing hormone (CRH), C-reactive protein (CRP), CD40 ligand, cortisol, epidermal growth factor (EGF), granulocyte colony-stimulating factor (G-CSF), interleukin-1 (IL-1), interleukin-1 Receptor Agonist (IL-1RA), interleukin-6 (IL-6), interleukin-10 (IL-10), interleukin-13 (IL-13), interleukin-18 (IL-18), leptin, macrophage inflammatory protein 1-alpha (MIP-1α), myeloperoxidase, neurotrophin 3 (NT-3), pancreatic polypeptide, plasminogen activator inhibitor-1, prolactin, RANTES, resistin, reelin (RELN), S100B, soluble tumor necrosis factor alpha (TNF-α), thyroid stimulating hormone (TSH), tumor necrosis factor alpha (TNF-α), or any combination thereof.

Individual analytes from the library can be evaluated for correlation to a particular clinical condition. As depicted in the flow diagram of FIG. 1, the process of developing a disease-specific panel of biomarkers can include two statistical approaches: 1) testing the distribution of analytes for association with the disease by univariate analysis; and 2) clustering the analytes into groups using multivariate analysis. In some cases, univariate analysis can be performed to test the distribution of biomarkers for association with MDD, and linear discriminant analysis (LDA) and binary logistic regression can be performed to construct an algorithm to generate a diagnostic score. Univariate analysis explores each variable in a data set separately and identifies the range and central tendency of the values. Multivariate analysis divides the variables into non-overlapping, uni-dimensional clusters. Two or more analytes from each cluster can be selected to design a biomarker or analyte panel for further analyses. The selection typically is based on the statistical strength of the markers and current biological understanding of the disease. For example, analytes chosen according to statistical significance can be subjected to multivariate analysis to identify markers which can distinguish subjects with a clinical condition such as depression from normal populations. Methods for determining statistical significance can be those routinely used in the art including, for example: t-statistics, chi-square statistics, and F-statistics.

In some cases, multivariate analysis can be linear discriminant analysis (LDA), a statistical method used to find the linear combination of features which best separate two or more classes of objects or events. In some cases, multivariate analysis can be principal components analysis (PCA), which is a statistical method that transforms data to a new coordinate system such that the greatest variance by any projection of the data comes to lie on the first coordinate (called the first principal component), the second greatest variance on the second coordinate, and so on. PCA can be used for dimensionality reduction in a data set by retaining those characteristics of the data set that contribute most to its variance, by keeping lower-order principal components and ignoring higher-order ones. Such low-order components often contain the “most important” aspects of the data. In some cases, multivariate analysis can be partial least squares discriminant analysis (PLS-DA), a statistical method used to maximize the separation between groups of variables by rotating PCA components such that a maximum separation among clusters is obtained, and to identify which variables distinguish and separate the clusters.

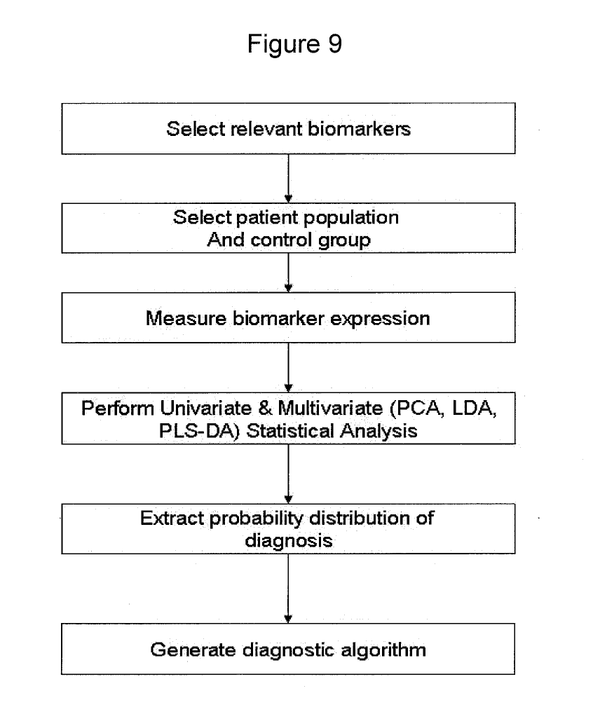

As depicted in the flow diagram of FIG. 9, the selection of relevant biomarkers need not be dependent upon the selection process described in FIG. 1, although the first process is efficient and can provide an experimentally and statistically based selection of markers. The process can be initiated, rather, by a group of biomarkers selected entirely on the basis of hypothesis and currently available data. The selection of a relevant patient population and appropriately matched (e.g., for age, sex, race, and/or BMI) population of normal subjects typically is involved in the process.

### Qualifying Biomarkers

This document also provides materials and methods for qualifying both disease related and pharmacodynamic biomarkers. A consistent framework for acceptance and qualification of biomarkers for regulatory use can facilitate innovative and efficient research and subsequent application of biomarkers in drug and therapeutic regimen development. Cumulative data (e.g., from multiple laboratories, perhaps a biomarker consortium model) may drive efficient execution of research and ultimately regulatory acceptance of biomarkers for specific indications. In the assessment of complex diseases including neuropsychiatric diseases such as MDD, as described herein, studies of well characterized patient and control normal subjects have been undertaken as part of a biomarker qualification process. Biomarker qualification is a graded, “fit-for-purpose” evidentiary process that links a biomarker with biology and with clinical end points. As clinical experience with biomarker panels is developed, information relevant to biomarker qualification and eventually regulatory acceptance of biomarkers also is developed for specific disease applications, as well as pharmacodynamic and efficacy markers.

Traditional cumulative clinical studies (e.g., assaying biological samples, clinical measures, imaging analysis) can be used in the qualification process. In some cases, biomarker expression can be measured in a statistically powered cohort of patients treated with an antidepressive treatment (e.g., TMS, VNS, or placebo (without magnetic or electrical pulse)). The age and sex of the cohort of patients can be adjusted to conform to the distribution of MDD patients in the general population. Such studies can reveal the possibility and nature of a placebo effect in therapy (e.g., TMS or VSN therapy). In the case of MDD, comparisons can be made between biomarkers with a positive response to placebo or a psychoactive substance (e.g., lithium) to positive changes observed in patients being treated with therapies such as antidepressant pharmaceuticals, electro-convulsive treatment (ECT), TMS, VNS, or cognitive behavioral therapy (CBT).

### Analyte Measurement

The methods of stratifying disease severity and monitoring a subject's response to treatment for depression provided herein can include determining the levels of a group of biomarkers in a biological sample collected from the subject. An exemplary subject is a human, but subjects can also include animals that are used as models of human disease (e.g., mice, rats, rabbits, dogs, and non-human primates). The group of biomarkers can be specific to a particular disease. For example, a plurality of analytes can form a panel specific to MDD.

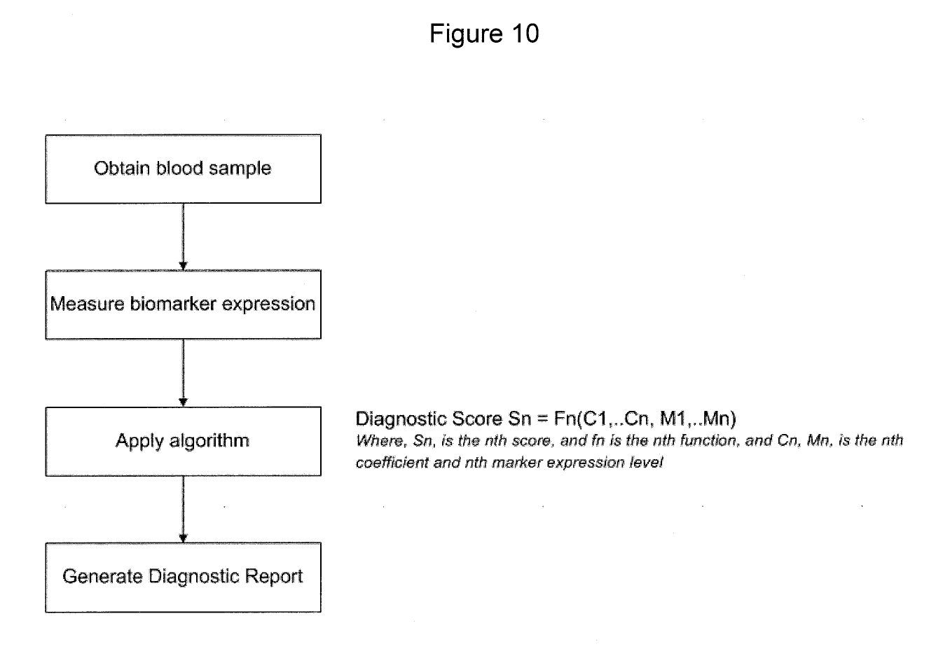

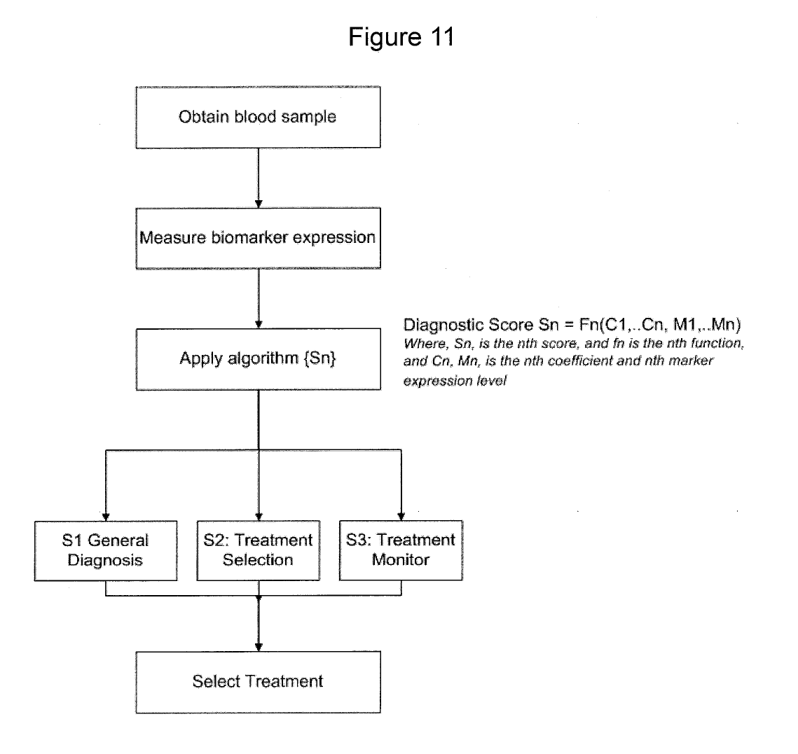

A number of suitable methods can be used to quantify the parameters included in a diagnostic/prognostic algorithm, and to quantify treatment-specific analyte expression. For example, measurements (e.g., analyte measurements) can be obtained using one or more medical devices or clinical evaluation scores to assess a subject's condition. As depicted in the flow diagram of FIG. 10, the methods provided herein for establishing a diagnostic score can include using tests of biological samples to determine the levels of particular analytes. As used herein, a “biological sample” is a sample that contains cells or cellular material, from which nucleic acids, polypeptides, or other analytes can be obtained. Depending upon the type of analysis being performed, the biological sample can be serum, plasma, or blood cells isolated by standard techniques. Serum and plasma are exemplary biological samples, but other biological samples can be used. For example, specific monoamines can be measured in urine. In addition, depressed patients as a group have been found to excrete greater amounts of catecholamines (CAs) and metabolites in urine than healthy control subjects. Examples of other suitable biological samples include, without limitation, cerebrospinal fluid, pleural fluid, bronchial lavages, sputum, peritoneal fluid, bladder washings, secretions (e.g., breast secretions), oral washings, swabs (e.g., oral swabs), isolated cells, tissue samples, touch preps, and fine-needle aspirates. In some cases, if the biological sample is to be tested immediately, the sample can be maintained at room temperature; otherwise the sample can be refrigerated or frozen (e.g., at −80° C.) prior to assay. In some cases, samples are collected from the subject at regular intervals following treatment (e.g., with TMS or VNS, or mock stimulation). In some cases, samples can be collected minutes, hours, days, or weeks following treatment (e.g., with TMS or VNS, or mock stimulation).

Measurements can be obtained separately for individual parameters, or can be obtained simultaneously for a plurality of parameters. Any suitable platform can be used to obtain parameter measurements. Multiplex methods of quantifying biomarkers can be particularly useful. An example of platform useful for multiplexing is the FDA-approved, flow-based LUMINEX® assay system (xMAP®; Luminex Corporation, Austin, Tex.). This multiplex technology uses flow cytometry to detect antibody/peptide/oligonucleotide or receptor tagged and labeled microspheres. In addition, LUMINEX® technology permits multiplexing of up to 100 unique assays within a single sample. Since the system is open in architecture, LUMINEX® can be readily configured to host particular disease panels.

Another useful technique for analyte quantification is immunoassay, a biochemical test that measures the concentration of a substance (e.g., in a biological tissue or fluid such as serum, plasma, cerebral spinal fluid, or urine) based on the specific binding of an antibody to its antigen. Antibodies chosen for biomarker quantification typically have a high affinity for their antigens. A vast array of different labels and assay strategies has been developed to meet the requirements of quantifying plasma proteins with sensitivity, accuracy, reliability, and convenience. For example, Enzyme Linked ImmunoSorbant Assay (ELISA) can be used to quantify biomarkers in a biological sample (e.g., serum or plasma). In a “solid phase sandwich ELISA,” an unknown amount of a specific “capture” antibody can be affixed to a surface of a multiwell plate, and the sample can be allowed to absorb to the capture antibody. A second specific, labeled antibody then can be washed over the surface so that it can bind to the antigen. The second antibody is linked to an enzyme, and in the final step a substance is added that can be converted by the enzyme to generate a detectable signal (e.g., a fluorescent signal). For fluorescence ELISA, a plate reader can be used to measure the signal produced when light of the appropriate wavelength is shown upon the sample. The quantification of the assay's endpoint involves reading the absorbance of the colored solution in different wells on the multiwell plate. A range of plate readers are available that incorporate a spectrophotometer to allow precise measurement of the colored solution. Some automated systems, such as the BIOMEK® 1000 (Beckman Instruments, Inc.; Fullerton, Calif.), also have built-in detection systems. In general, a computer can be used to fit the unknown data points to experimentally derived concentration curves.

In some cases, biomarker (e.g., analyte) expression levels in a biological sample can be measured using a mass spectrometry instrument (e.g., a multi-isotope imaging mass spectrometry (MIMS) instrument), or any other suitable technology, including, for example, technology for measuring expression of RNA. Such methods include, for example, PCR and quantitative real time PCR methods using a dual-labelled fluorogenic probe (e.g., TAQMAN™, Applied Biosystems, Foster City, Calif.). In some cases, DNA microarrays can be used to study gene expression patterns on a genomic scale. Microarrays allow for simultaneous measurement of changes in the levels of thousands of messenger RNAs within a single experiment. Microarrays can be used to assay gene expression across a large portion of the genome prior to, during, and/or after a treatment regimen. The combination of microarrays and bioinformatics can be used to identify biomolecules that are correlated to a particular treatment regimen or to a positive or negative response to treatment. In some cases, microarrays can be used in conjunction with proteomic analysis.

Useful platforms for simultaneously quantifying multiple protein parameters include, for example, those described in U.S. Provisional Application Nos. 60/910,217 and 60/824,471, U.S. Utility application Ser. No. 11/850,550, and PCT Publication No. W02007/067819, all of which are incorporated herein by reference in their entirety. An example of a useful platform utilizes MIMS label-free assay technology developed by Precision Human Biolaboratories, Inc. (now Ridge Diagnostics, Inc., Research Triangle Park, N.C.). Briefly, local interference at the boundary of a thin film can be the basis for optical detection technologies. For biomolecular interaction analysis, glass chips with an interference layer of SiO₂ can be used as a sensor. Molecules binding at the surface of this layer increase the optical thickness of the interference film, which can be determined as set forth in the applications listed above, and in U.S. Provisional Application Nos. 60/910,217 and 60/824,471, for example.

With regard to the potential for new biomarker discovery, traditional 2-dimensional gel electrophoresis can be performed for protein separation, followed by mass spectrometry (e.g., MALDI-TOF, MALDI-ESI) and bioinformatics for protein identification and characterization. Other methods of differential protein quantification can be used. For example, tandem mass spectrometry (MS/MS) can be used to simultaneously determine both the identity and relative abundances of proteins and peptides.

### Transcranial Magnetic Stimulation and Vagus Nerve Stimulation

An advantage of using TMS or VNS as opposed to antidepressant drugs in assessing physiological changes related to treatment efficacy is that TMS and VNS treatment are of brief duration and are physical rather than biochemical in nature.

This document provides methods for determining a subject's diagnostic scores pre- and post-TMS. TMS is a noninvasive technique used to treat neuropsychiatric diseases such as major depression, schizophrenia, mania, post-traumatic stress disorder, Tourette's disorder, Parkinson's disease, and obsessive compulsive disorder. TMS involves discharging electrical energy through a conducting coil to produce a transient magnetic field that causes an electrical current to flow to a secondary conducting material such as neuronal tissue. Since the scalp and skull are largely nonconductive, the transient magnetic field penetrates these tissues to target specific cortical regions of the brain. Stimulation of the frontal cortex has been demonstrated to induce short- and long-term changes in behavior and mood in healthy subjects and subjects with MDD. For review, see Paus and Barrett, J. Psychiatry Neurosci. 29:268-79 (2004).

A number of methods of administering TMS can be used. An exemplary protocol can be found at neuronetics.com on the World Wide Web. TMS can be administered using either a biphasic or monophasic magnetic pulse. A biphasic pulse is sinusoidal and is generally of shorter duration than a monophasic pulse, which involves a rapid rise from zero followed by a slow decay back to zero. In addition, TMS can be administered using either circular or figure eight-shaped conductive coils. While circular coils are generally more powerful, figure eight-shaped coils produce a more focused magnetic field and a better spatial resolution of activation. An antidepressant effect often is evident at a range (e.g., 1-25 Hz) of frequencies. Both the orientation and intensity of the conductive coil determine the type of tissue stimulated and the strength of that stimulation. In some cases, TMS can be repetitive TMS (rTMS), in which a train of magnetic pulses are administered to a subject. Repetitive TMS using varying frequencies and intensities can increase or decrease excitability in a cortical area directly targeted by the stimulation. For example, the left prefrontal cortex is less active in subjects with clinical depression, and the prefrontal cortex is readily accessible to TMS. Mock stimulation can be used as a control or placebo for TMS or rTMS. The NeuroStar TMS Therapy system (neuronetics.com on the World Wide Web) is an example of an FDA-approved TMS Therapy® device that can be used for treatment of depression and in biomarker studies.

This document also provides methods for determining a subject's diagnostic scores pre- and post-VNS. VNS is a minimally invasive technique used to treat neuropsychiatric diseases such as, for example, major depression (e.g., treatment-resistant depression) and bipolar disorder. VNS involves delivering intermittent electrical stimulation to a vagus nerve from an implanted pacemaker-like pulse generator and a nerve stimulation electrode. For example, an implantable device can be programmed to deliver mild, intermittent electrical pulses to the left vagus nerve. Stimulation of the left vagus nerve can induce short- and long-term changes in behavior and mood in healthy subjects and in subjects with MDD. For review, see Park et al., Acta Neurochir Suppl. 97:407-16 (2007).

A number of methods of administering VNS can be used. An exemplary protocol can be found at vnstherapy.com on the World Wide Web. VNS can be administered using an on/off stimulation cycle. In some cases, a stimulation cycle can be 30 seconds of electrical stimulation (an “on” phase) followed by 5 minutes of no electrical stimulation (an “off” phase). An exemplary set of stimulation parameters can include: an output current of 1 mA, a frequency of 20 Hz, a pulse width of 500 μsec, an “on” phase of 30 seconds, and an “off' phase of 5 minutes. In some cases, the output current can range from about 0 to about 2.25 mA. In some cases, the frequency can range from about 2 to about 30 Hz (e.g., about 2, about 5, about 10, about 15, about 20, about 25, or about 30 Hz). In some cases, the pulse width can range from about 130 to about 750 μsec (e.g., about 130, about 150, about 200, about 250, about 300, about 350, about 400, about 450, about 500, about 550, about 600, about 650, about 700, or about 750 μsec). In some cases, the “on” phase can range from about 7 to about 60 seconds, and the “off” phase can range from about 0.3 minutes to 180 minutes (e.g., about 0.3, about 0.5, about 1, about 2, about 5, about 10, about 20, about 30, about 40, about 50, about 60, about 90, about 120, about 150, or about 180 minutes). A pulse-generating implantable device can be reprogrammed to alter the stimulation cycle. Mock stimulation can be used as a control or placebo for VNS. The VNS Therapy™ Pulse Model 102R Generator system and the VNS Therapy™ Pulse Duo Model 102R Generator system (Cyberonics, Inc., Houston, Tex.) are examples of FDA-approved pulse-generating devices that can be used for treatment of depression and in biomarker studies. Such devices can be used in conjunction with a bipolar electrical lead that transmits stimulation from the pulse-generating device to the left vagus nerve of a subject. Any appropriate method can be used to implant a pulse-generating device and/or electrical leads for VNS. For example, a device for VNS can be implanted in a subject under general anesthesia in an outpatient procedure. In some cases, implantation can be performed according to methods used to place pulse-generating devices in subjects having epilepsy.

### Use of Biomarker Hypermapping (BHYPERMAP ™)

This document also provides methods for using biomarker hypermapping to evaluate patients pre- and post-TMS or post-VNS. This approach uniquely includes the construction of a multianalyte hypermap versus analyzing single markers either alone or in groups. Biomarker hypermapping uses multiple markers from a human biomarker collection and interrelated algorithms to distinguish individual groups of patients. Using clusters of biomarkers reflective of different physiologic parameters (e.g., hormones vs. inflammatory markers), a patient's biomarker responses can be mapped onto a multi-dimensional hyperspace. As described herein, four classes of biomarkers are used in the process of mapping changes in response to therapy:

Inflammatory biomarkers

HPA axis biomarkers

Metabolic biomarkers

Neurotrophic biomarkers

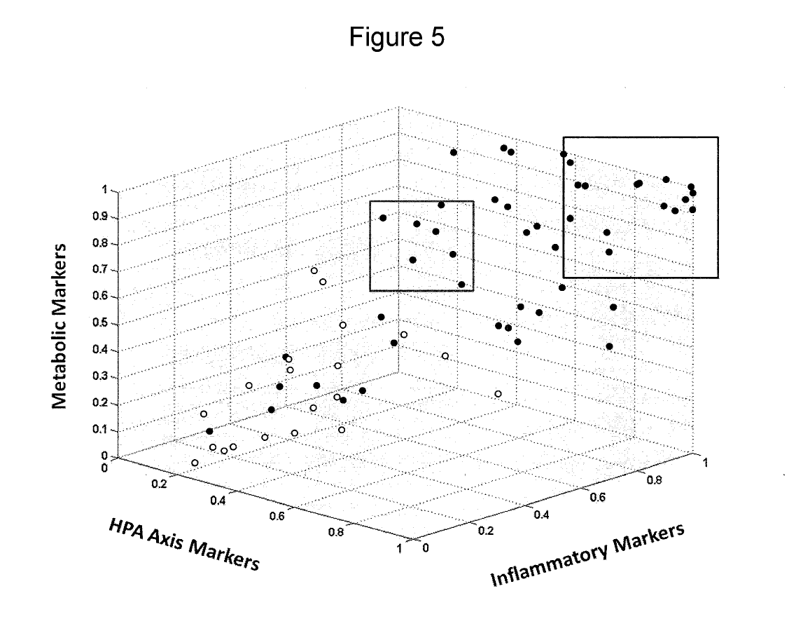

Four vectors can be created for the four classes of biomarkers; together, the vectors form a point in a hyperspace. A computer program can be used to analyze the data, plot the vectors, and populate the hypermap. For ease of visualization, a three-dimensional hypermap can be created using vectors established from three of the four classes of physiologically defined biomarkers. This initially can be done for a patient at the time s/he is first tested, to aid in their classification. FIG. 5 illustrates the concept. Distinct coefficients were used to create hyperspace vectors for 50 MDD patients and 20 age-matched normal subjects. Multiplex biomarker data from clinical samples were used to display individual patients (filled circles) and normal subjects (open circles) on a hyperspace map where the axes are HPA axis, inflammatory and metabolic markers. Unlike the MDD score that provides a numerical value for the patient, the hypermap discloses information relative to the expression of different classes of markers. By way of example, the patients in the small square have higher values for metabolic and inflammatory markers, while those in the larger rectangle have high values for HPA axis markers in addition to the two other marker groups. As clinically relevant information (e.g., disease severity) is collected on increasingly larger numbers of patients, this technology may be an even more potent aid to patient management.

Further, a hypermap can, by addition of data on patient response, answer questions about preferred treatment regimens and assessment of treatment efficacy. By way of example, using a hypermap that incorporates a large amount of patient data surrounding biomarker changes and clinical response to a selective serotonin reuptake inhibitor (SSRI), areas of hyperspace (patterns) associated with an enhanced response to TMS or VNS vs. LEXAPRO™ [a serotonin and norepinephrin reuptake inhibitor (SNRI)] can be identified.

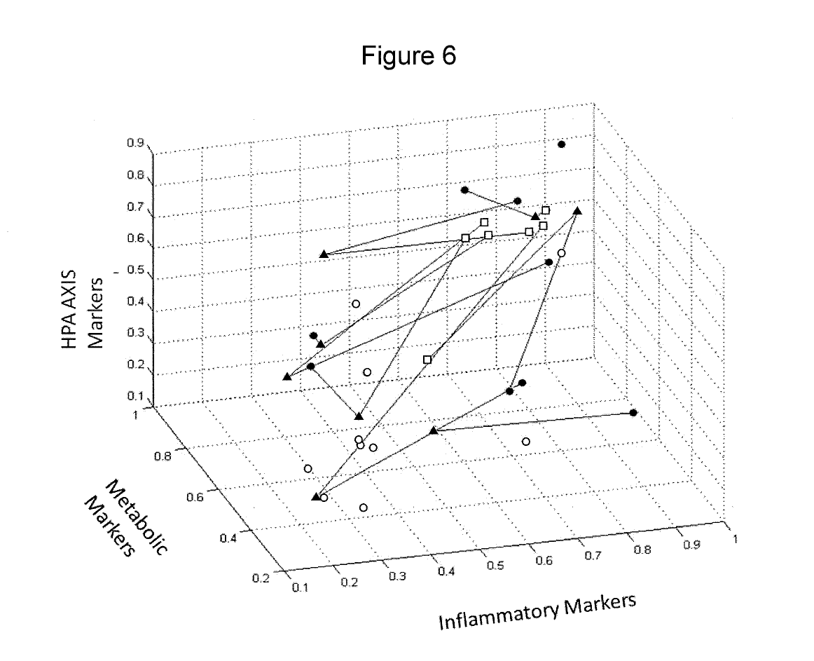

FIG. 6 shows a specific example of a biomarker hypermap indicating positive or negative response to treatment for a series of patients treated with LEXAPRO™. MDD patients at baseline are indicated by filled circles. Filled triangles represent patients after 2-3 weeks of treatment, and open squares represent patients after 8 weeks of treatment. Open circles represent untreated normal subjects.

### Computer-Based Systems

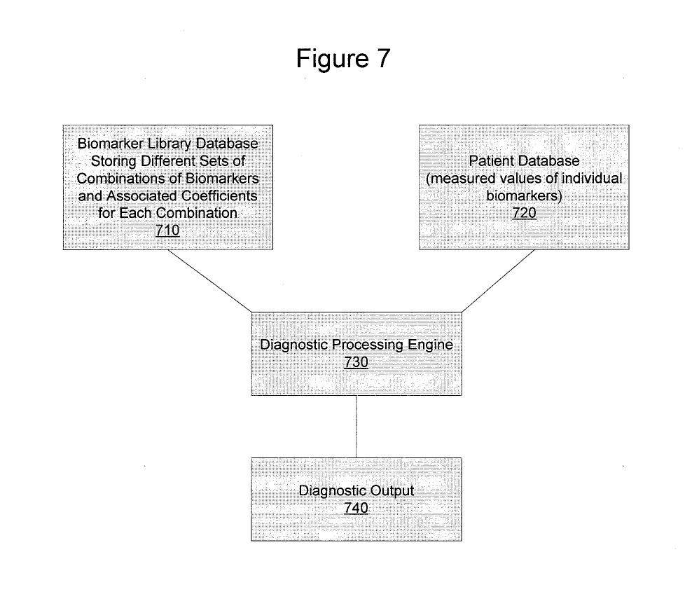

FIG. 7 shows an example of a computer-based diagnostic system employing the biomarker analysis described herein. This system includes a biomarker library database 710 that stores different sets combinations of biomarkers and associated coefficients for each combination based on biomarker algorithms which are generated based on, e.g., the methods described herein. The database 710 is stored in a digital storage device in the system. A patient database 720 is provided in this system to store measured values of individual biomarkers of one or more patients under analysis. A diagnostic processing engine 730, which can be implemented by one or more computer processors, is provided to apply one or more sets of combinations of biomarkers in the biomarker library database 710 to the patient data of a particular patient stored in the database 720 to generate diagnostic output for a set of combination of biomarkers that is selected for diagnosing the patient. Two or more such sets may be applied to the patient data to provide two or more different diagnostic output results. The output of the processing engine 730 can be stored in an output device 740, which can be, e.g., a display device, a printer, or a database.

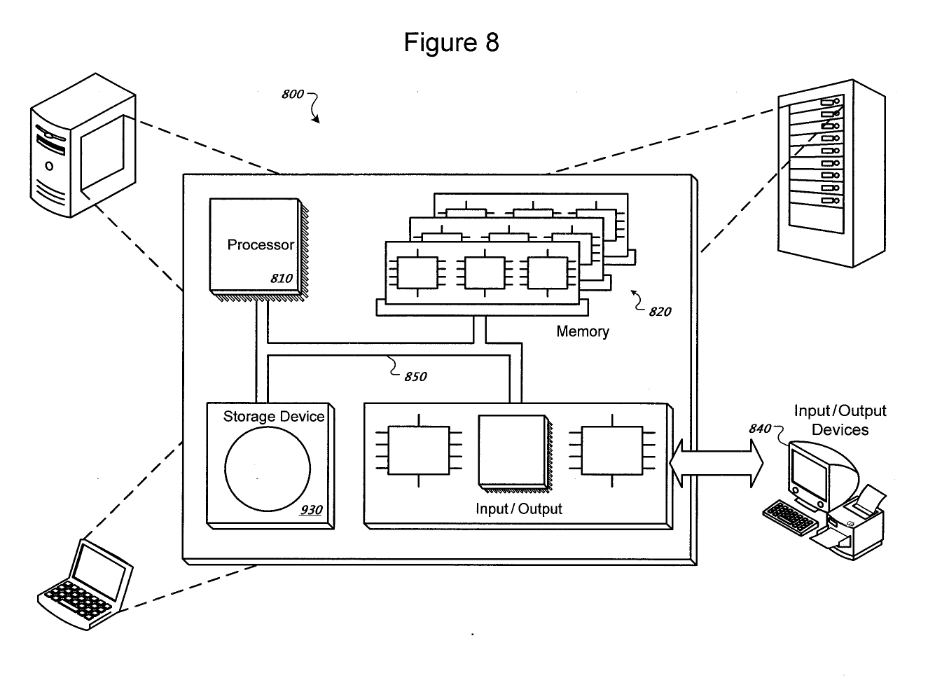

One or more computer systems can be used to implement the system in FIG. 7 and for the operations described in association with any of the computer-implement methods described in this document. FIG. 8 shows an example of such a computer system 800. The system 800 can include various forms of digital computers, such as laptops, desktops, workstations, personal digital assistants, servers, blade servers, mainframes, and other appropriate computers. The system 800 can also include mobile devices, such as personal digital assistants, cellular telephones, smartphones, and other similar computing devices. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

In the specific example in FIG. 8, the system 800 includes a processor 810, a memory 820, a storage device 830, and an input/output device 840. Each of the components 810, 820, 830, and 840 are interconnected using a system bus 850. The processor 810 is capable of processing instructions for execution within the system 800. The processor may be designed using any of a number of architectures. For example, the processor 810 may be a CISC (Complex Instruction Set Computers) processor, a RISC (Reduced Instruction Set Computer) processor, or a MISC (Minimal Instruction Set Computer) processor.

In some embodiments, the processor 810 is a single-threaded processor. In other embodiments, the processor 810 is a multi-threaded processor. The processor 810 is capable of processing instructions stored in the memory 820 or on the storage device 830 to display graphical information for a user interface on the input/output device 840.

The memory 820 stores information within the system 800. In some embodiments, the memory 820 is a computer-readable medium. In other embodiments, the memory 820 is a volatile memory unit. In still other embodiments, the memory 820 is a non-volatile memory unit.

The storage device 830 is capable of providing mass storage for the system 800. In some embodiments, the storage device 830 is a computer-readable medium. In various different embodiments, the storage device 830 may be a floppy disk device, a hard disk device, an optical disk device, or a tape device.

The input/output device 840 provides input/output operations for the system 800. In some embodiments, the input/output device 840 includes a keyboard and/or pointing device. In some cases, the input/output device 840 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment. For example, a computer program can use biomarker measurements for an MDD patient's set of biomarker pathways (e.g., inflammation, metabolic, neurotrophic, or HPA axis) to calculate vectors and position the patient's data on a hypermap of other patients treated with TMS or VNS.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

### Methods for Using Diagnostic Scores

Diagnostic scores and pharmacodynamic biomarkers can be used for, without limitation, treatment monitoring. For example, as depicted in the flow diagram of FIG. 11, diagnostic scores can be used to aid in determining diagnosis, stratifying patients, selecting treatments, and monitoring treatment. One or more multiple diagnostic scores can be generated from the expression levels of a set of biomarkers. In this example, multiple biomarkers can be measured from a subject's blood sample, generating three diagnostic scores by the algorithm. In some cases, a single diagnostic score can be sufficient to aid in making a diagnosis and selecting treatment.

Diagnostic scores generated by the methods provided herein can be used to, for example, stratify disease severity. Thus, in some cases, individual analyte levels and/or diagnostic scores determined by the algorithms provided herein can be provided to a clinician for use in diagnosing a subject as having mild, moderate, or severe depression. For example, diagnostic scores generated using the algorithms provided herein can be communicated by research technicians or other professionals who determine the diagnostic scores to clinicians, therapists, or other health-care professionals who will classify a subject as having a particular disease severity based on the particular score, or an increase or decrease in diagnostic score over a period of time. On the basis of these classifications, clinicians, therapists, or other health-care professionals can evaluate and recommend appropriate treatment options, educational programs, and/or other therapies with the goal of optimizing patient care. Diagnoses can be made, for example, using state of the art methodology, or can be made by a single physician or group of physicians with relevant experience with the patient population.

As described herein, a method can include providing a numerical value for each of a plurality of analytes relevant to mild, moderate, and severe states of depression, wherein each numerical value corresponds to the level of the analyte in a biological sample from the subject individually weighting each numerical value in a manner specific to each analyte to obtain a weighted value for each analyte, and determining a result value based on an equation that includes each weighted value. The result value can then be compared to control result values (e.g., values obtained using biological samples from normal subjects and from subjects having mild, moderate, and severe depression), provided that the control result values were determined in a manner comparable to that for the result value. The subject can then be classified as not having depression or as having mild depression, moderate depression, or severe depression, based on where the result value falls as compared to the control values. The method can include using an algorithm to calculate a MDD diagnostic score that can be used to support the classification. The plurality of analytes can include any two or more of those listed in Table 5 herein. In some cases, the plurality can include cortisol, prolactin, EGF, MPO, BDNF, resistin, sTNFR2, ApoC3, and A1AT, for example. In addition, the method can include obtaining a measured level of one or more of the plurality of analytes for the biological sample, wherein the result value is based at least in part on the measured level.

Diagnostic scores also can be used for treatment monitoring. For example, diagnostic scores and/or individual analyte levels can be provided to a clinician for use in establishing or altering a course of treatment for a subject. When a treatment is selected and treatment starts, the subject can be monitored periodically by collecting biological samples at two or more intervals, measuring biomarker levels to determine a diagnostic score corresponding to a given time interval (e.g., pre- and post-treatment), and comparing diagnostic scores over time. On the basis of these scores and any trends observed with respect to increasing, decreasing, or stabilizing diagnostic scores or changes in pharmacodynamic biomarker levels, a clinician, therapist, or other health-care professional may choose to continue treatment as is, to discontinue treatment, or to adjust the treatment plan with the goal of seeing improvement over time. For example, a decrease in disease severity as determined by a change in diagnostic score can correspond to a patient's positive response to treatment. An increase in disease severity as determined by a change in diagnostic score can indicate failure to respond positively to treatment and/or the need to reevaluate the current treatment plan. In some embodiments, an increase in the level of a pharmacodynamic biomarker that correlates to positive responses to a particular treatment regimen for neuropsychiatric disease can indicate a patient's positive response to treatment, and a decrease in the level of such a pharmacodynamic biomarker can indicate failure to respond positively to treatment and/or the need to reevaluate the current treatment plan. A static diagnostic score can correspond to stasis with respect to disease severity. The biomarker pattern may be different for patients who are on antidepressants or are undergoing other forms of therapy (e.g., treatment with a specific antidepressant, cognitive behavioral therapy (CBT) or electro-convulsive therapy (ECT)) in addition to TMS or VNS for example, and changes in the diagnostic score toward that of normal patients can be an indication of an effective therapy combination. As the cumulative experience with therapies increases, specific biomarker panels can be derived to monitor responses to treatment (e.g., with TMS or VNS) in combination with therapy with specific antidepressants, etc. In some cases, movement between disease strata (i.e., mild, moderate, and severe depression) can indicate increasing or decreasing disease severity. In some cases, movement between disease strata can correspond to efficacy of the treatment plan selected for a particular subject or group of subjects.

In some cases, a method can include (a) providing a first numerical value for each of a plurality of analytes relevant to depression, wherein each first numerical value corresponds to the level of the analyte in a first biological sample from the subject, individually weighting each first numerical value in a manner specific to each analyte to obtain a first weighted value for each analyte, and determining a first MDD score based on an equation that includes each first weighted value; and (b) providing a second numerical value of each of the plurality of analytes, wherein each second numerical value corresponds to the level of the analyte in a second biological sample from the subject, wherein the second biological sample is obtained after treatment for the depressive disorder (e.g., treatment for days, weeks, months, or more), individually weighting each second numerical value in a manner specific to each analyte to obtain a second weighted value for each analyte, with the proviso that the weighting is done in a manner comparable to that in step (a), and using the equation to determine a second MDD score after treatment of the subject for the depressive disorder. The first MDD score can be compared to the second MDD score and to a control MDD score or range of MDD scores determined from one or more normal subjects, and the treatment can be classified as effective if the second MDD score is closer than the first MDD score to the control MDD score, or classifying as not effective if the second MDD score is not closer than the first MDD score to the control MDD score.

In some embodiments, an initial blood sample is taken from a subject prior to the start of treatment. The sample optionally can be spun down to separate serum from cells, and stored as PS1 (Patient p draw 1). The subject then can be treated (e.g., with one or more antidepressant drugs) for a length of time, and blood samples can be collected during the course of treatment (e.g., days, weeks, or months after the beginning of treatment). The samples optionally can be spun down, labeled and stored, such that together with the initial sample, there can be multiple samples - PS1, PS2, PS3, etc., depending on the duration of treatment and the frequency of sample collection.

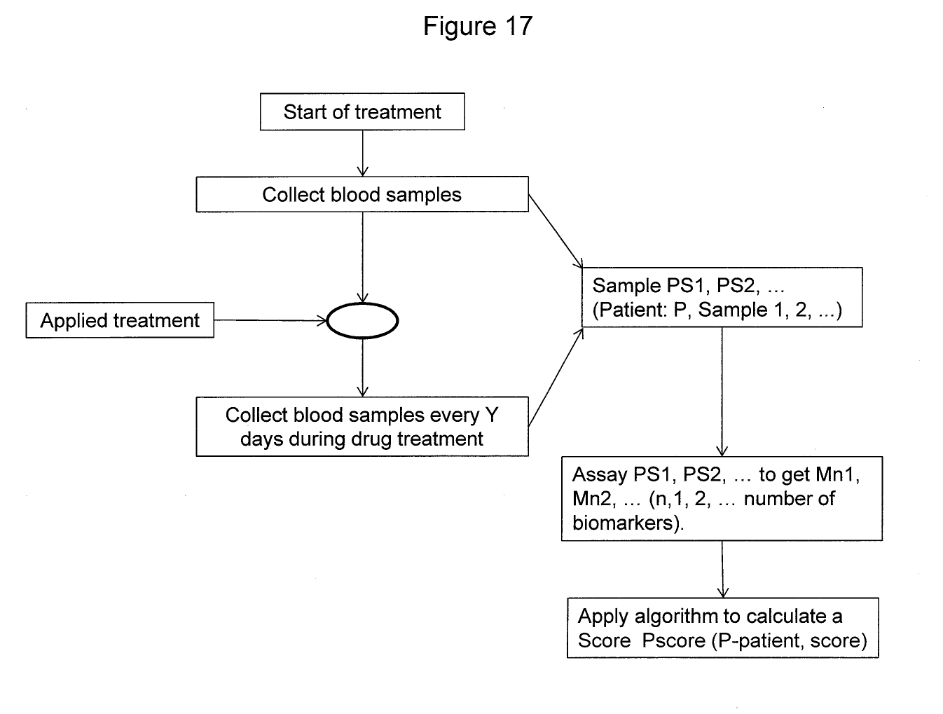

The samples (PS1, PS2, PS3, etc.) for the subject can be assayed to measure the levels of selected biomarkers (Mn1, Mn2, and Mn3 =biomarkers n1, n2, and n3; see, e.g., FIG. 17). The marker panel can include biomarkers selected from four major biological systems/pathways (inflammation, HPA axis, metabolic biomarkers, and neurotrophic factors, as described herein). For example, the biomarkers in each pathway include a selection of biomarkers from the list shown in Table 1 (e.g., RANTES, PRL, BDNF, S100B, RES, TNFR, A1A, cortisol, and EGF; RANTES, PRL, BDNF, S100B, RES, TNFR, A1A, and EGF; RANTES, PRL, BDNF, S100B, RES, TNFR, and A1A; S100B, PRL, BDNF, RES, TNFR, and A1A; cortisol, PRL, BDNF, RES, TNFR, and A1A; or BDNF, resistin, TNFRII, and A1A).

A mathematical algorithm can be applied the biomarker measurements to calculate a score that is correlated to the final outcome (e.g., the HAMD score change) at the end of the antidepressant treatment period. The mathematical algorithm can use the specific biomarker changes and the rates of those changes to calculate the score. For example, Mn1, Mn2 (n=1, 2, . . . number of markers) can be used to calculate the score. Alternatively, the changes of (Mn1−Mn2(Mn1+Mn2) can be used to calculate the score if only two samples are used. In another example, Mn1,Mn2, Mn3 (n=1, 2, 3, . . . number of markers) can be used to calculate the score, and if three samples are collected, the changes of (Mn1−Mn2)/(Mn1+Mn2), (Mn2−Mn3)/(Mn2+Mn3) can be used to calculate the score.

At the end of treatment, the outcome for each patient is known (i.e., whether treatment is successful). This result can be used as an input to optimize the calculation that includes using biomarker measurements (Mn1, Mn2, Mn3, etc.) to predict patient treatment results. Comparing the clinical outcome with the biomarker measurements can optimize generation of a score that maximally correlates to the treatment outcome for a patient treated for depression (e.g., with an antidepressant drug). A control monitoring score can be determined using such a method, and the control score subsequently can be used as a standard to ascertain whether treatment of a subject for depression is effective.

In other embodiments, a method can include at least two (e.g., two, three, four, five, or more than five) numerical values for each of a plurality of analytes relevant to depression, wherein each numerical value corresponds to the level of the analyte in a biological sample from the subject. For example, a first numerical value can be obtained for an analyte in a first biological sample obtained from the subject, a second numerical value can be obtained for the analyte in a second biological sample from the subject, etc. The first biological sample can be obtained before treatment for the depressive disorder, and the second and any subsequent biological samples can be obtained after the onset of treatment (e.g., 12 hours after treatment onset, or one, two, three, four, five, six, seven, 14, 21, or more days after treatment onset). The numerical values can be individually weighted in a manner specific to each analyte, thus giving a weighted value for each analyte, and a “monitoring score” can be determined based on an equation that includes the weighted numerical values. The monitoring score can be compared to a control score, and the success of the treatment can be gauged based on whether the calculated monitoring score is greater than the control score. For example, treatment can be classified as being effective if the monitoring score is greater than or equal to the control monitoring score, or classified as not being effective if the monitoring score is less than the control monitoring score. The plurality of analytes can include any two or more of those listed in Table 5 herein. For example, the plurality of analytes can include PRL, BDNF, RES, TNFRII, and A1A; or RANTES, PRL, BDNF, S100B, RES, TNFR, A1A, and EGF. The control value can be determined from a clinical treatment monitoring study, such that trial data is used to determine a monitoring score that correlates with treatment outcome (e.g., successful treatment). That monitoring score can be established as the control.

After a patient's diagnostic scores are reported, a health-care professional can take one or more actions that can affect patient care. For example, a health-care professional can record a diagnostic or monitoring score, and/or biomarker expression levels, in a patient's medical record. In some cases, a health-care professional can record a diagnosis of a neuropsychiatric disease (e.g., MDD), or otherwise transform the patient's medical record, to reflect the patient's medical condition. In some cases, a health-care professional can review and evaluate a patient's medical record, and can assess multiple treatment strategies for clinical intervention of a patient's condition.

For major depressive disorder and other mood disorders, treatment monitoring can help a clinician adjust treatment dose(s) and duration. An indication of a subset of alterations in individual biomarker levels that more closely resemble normal homeostasis can assist a clinician in assessing the efficacy of a regimen. A health-care professional can initiate or modify treatment for symptoms of depression (e.g., MDD) and other neuropsychiatric diseases after receiving information regarding a patient's diagnostic score. In some cases, previous reports of diagnostic scores and/or individual biomarker (e.g., analyte) levels can be compared with recently communicated diagnostic scores and/or disease states. On the basis of such comparison, a health-care profession may recommend a change in therapy. In some cases, a health-care professional can enroll a patient in a clinical trial for novel therapeutic intervention of MDD symptoms. In some cases, a health-care professional can elect waiting to begin therapy until the patient's symptoms require clinical intervention.

A health-care professional can communicate diagnostic scores and/or individual biomarker (e.g., analyte) levels to a patient or a patient's family. In some cases, a health-care professional can provide a patient and/or a patient's family with information regarding MDD, including treatment options, prognosis, and referrals to specialists, e.g., neurologists and/or counselors. In some cases, a health-care professional can provide a copy of a patient's medical records to communicate diagnostic scores and/or disease states to a specialist.

A research professional can apply information regarding a subject's diagnostic scores, biomarker levels, and/or disease states to advance MDD research. For example, a researcher can compile data on MDD diagnostic scores with information regarding the efficacy of a drug for treatment of MDD symptoms to identify an effective treatment. In some cases, a research professional can obtain a subject's diagnostic scores and/or individual biomarker (e.g., analyte) levels to evaluate a subject's enrollment or continued participation in a research study or clinical trial. A research professional can classify the severity of a subject's condition based on the subject's current or previous diagnostic scores. In some cases, a research professional can communicate a subject's diagnostic scores and/or individual biomarker (e.g., analyte) levels to a health-care professional, and/or can refer a subject to a health-care professional for clinical assessment of MDD and treatment of MDD symptoms.

Any appropriate method can be used to communicate information to another person (e.g., a professional), and information can be communicated directly or indirectly. For example, a laboratory technician can input diagnostic scores and/or individual analyte levels into a computer-based record. In some cases, information can be communicated by making a physical alteration to medical or research records. For example, a medical professional can make a permanent notation or flag a medical record for communicating a diagnosis to other health-care professionals reviewing the record. Any type of communication can be used (e.g., mail, e-mail, telephone, facsimile, and face-to-face interactions). Secure types of communication (e.g., facsimile, mail, and face-to-face interactions) can be particularly useful. Information also can be communicated to a professional by making that information electronically available (e.g., in a secure manner) to the professional. For example, information can be placed on a computer database such that a health-care professional can access the information. In addition, information can be communicated to a hospital, clinic, or research facility serving as an agent for the professional. The Health Insurance Portability and Accountability Act (HIPAA) requires information systems housing patient health information to be protected from intrusion. Thus, information transferred over open networks (e.g., the internet or e-mail) can be encrypted. When closed systems or networks are used, existing access controls can be sufficient.

The invention will be further described in the following examples, which do not limit the scope of the invention described in the claims.

### EXAMPLES

### Example 1

### Identification of Pharmacodynamic Biomarkers Associated with MDD

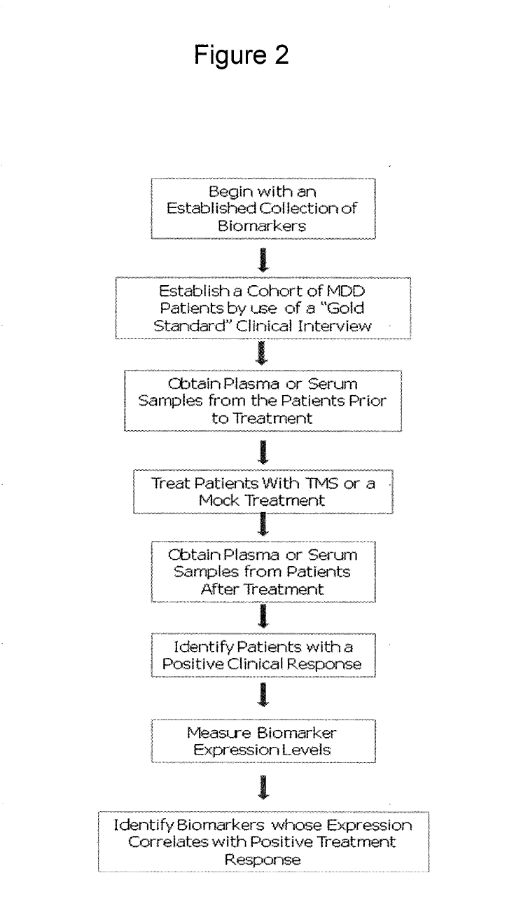

FIG. 2 illustrates a process of identifying pharmacodynamic biomarkers for MDD. A collection of biomarkers that have a potential association with MDD is selected based on the result of earlier studies, from a literature search, from genomic or proteomic analysis of biological pathways, or from molecular imaging studies. A cohort of MDD patients are identified using a “gold standard” method of interview-based clinical assessment. Plasma or serum samples are collected from each patient. Patients are then subjected to TMS or VNS stimulation or mock stimulation (placebo). Post-treatment plasma or serum samples are collected from each patient over a period of time (e.g., minutes, hours, days, and/or weeks after treatment). Expression levels of the selected biomarkers are measured for each sample. The patient's response to treatment, as determined by conducting additional structured clinical interviews and assigning post-TMS or post-VNS diagnostic scores, is recorded. Patients demonstrating a positive clinical response to TMS or VNS, which is defined as an improved post-treatment diagnostic score relative to the pre-treatment baseline score, are identified. Analytes whose expression correlates with positive clinical outcomes are identified as pharmacodynamic biomarkers for MDD.

Diagnostic biomarkers for MDD were generated using the steps outlined in FIG. 1, and a panel of about 20 analytes was established. These analytes included alpha-2-macroglobin (A2M), brain-derived neurotrophic factor (BDNF), C-reactive protein (CRP), cortisol, epidermal growth factor (EGF), interleukin 1 (IL-1), interleukin-6 (IL-6), interleukin-10 (IL-10), interleukin-18 (IL-18), leptin, macrophage inflammatory protein 1-alpha (MIP-1α), myeloperoxidase, neurotrophin 3 (NT-3), plasminogen activator inhibitor-1 (PAI-1), Prolactin (PRL), RANTES, resistin, S100B protein, soluble tumor necrosis factor alpha receptor type 2 (sTNFαRII), and tumor necrosis factor alpha (TNFα). These biomarkers or any combination thereof can be used for MDD diagnosis, stratification of patients for clinical trials, and/or patient monitoring.

### Example 2

### Using Proteomics to Analyze Multiple Biomarkers

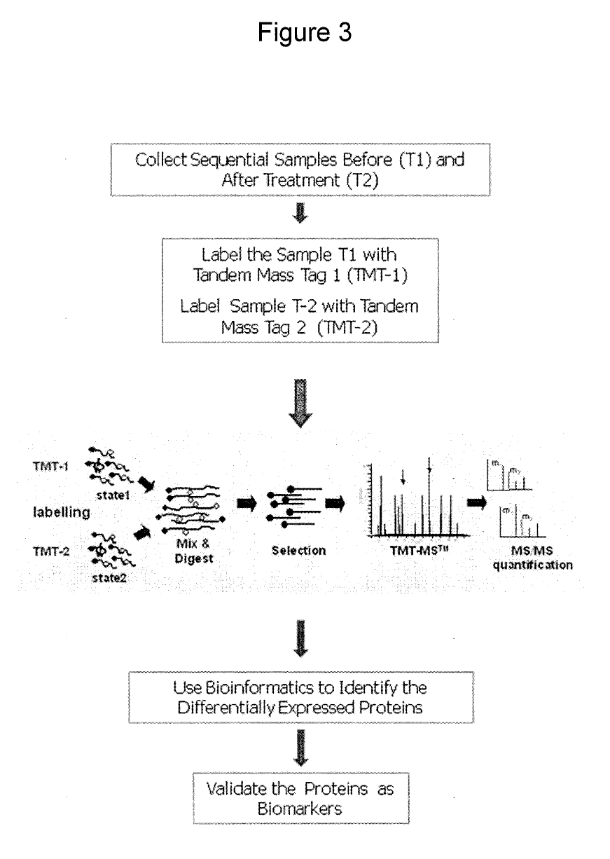

As shown in FIG. 3, treatment-relevant biomarkers are identified using tandem mass spectrometry. Biological samples are collected pre- and post-treatment. The samples are labeled with different Tandem Mass Tags (TMT) and mixed for TMT-MS™(Proteome Sciences, United Kingdom). Following fragmentation/digestion with a suitable enzyme (e.g., trypsin), TMT labeled fragments are selected for analysis by liquid chromatography MS/MS. The ratio of protein expression between samples is revealed by MS/MS by comparing the intensities of the individual reporter group signals. Bioinformatic analysis is used to determine the proteins that are differentially expressed. The identified proteins are then validated as potential biomarkers (e.g., using specific antibodies, and ELISA) over a defined period of time after treatment to establish a subset of pharmacodynamic biomarkers. Statistical analysis of a subject's changes in analyte expression levels is performed to correlate analytes with treatment efficacy. Upon statistical evaluation where statistical significance is defined as p<0.05, biomarkers having a p value greater than 0.05 are selected as biomarkers associated with therapy-responsive MDD.

### Example 3

### Using MDDSCORE™ and HAM-D scores to monitor treatment

An example of how MDDSCORE™ and HAM-D Scores can be used to monitor treatment-induced changes is shown in FIG. 4. The Hamilton Rating Scale for Depression (HAM-D) is a multiple choice questionnaire that clinicians often use to rate the severity of a patient's major depression. A HAM-D score greater than 18 was used as a cut off for MDD patients, based upon findings that trials initiated with higher mean baseline HAM-D scores were associated with greater reductions in HAM-D scores (a lower score indicates a reduction in severity) at the end of a 4- to 8-week trial than trials with a lower mean baseline HAM-D. In this example, Korean normal subjects (n=8, open circles in FIG. 4) and MDD patients who were drug naïve (n=8, filled circles) were evaluated by HAM-D at baseline only or baseline and after two weeks of treatment with LEXAPRO™ (open squares) respectively. Clinical results were obtained from serum samples from each of the eight MDD patients and eight normal subjects. The serum levels for each of the markers making up the MDDSCORE™ were determined by quantitative immunoassay. MDDSCORE™ was calculated, and the resultant data were graphed as the probability of having MDD on the x axis and HAM-D score on the y axis. MDDSCORE™ and HAM-D scores for patients treated with LEXAPRO™ for two weeks are indicated as open squares, and each is linked by an arrow to the same patient's value at baseline. The arrows indicate the directionality of change from prior to treatment of MDD patients (filled circles) and after two weeks of LEXAPRO™ treatment. For six of the eight MDD patients, both HAM-D score and MDDSCORE™ went down after treatment.

### Example 4

### Using Biomarker Hypermapping to Monitor Treatment

Clinical results were obtained from serum samples from 50 MDD patients and 20 normal subjects. The serum levels of each of the markers (listed below) were determined by quantitative immunoassay. A binary logistic regression optimization was used to fit the clinical data with selected markers in each group against the clinical results from the “gold standard” clinical evaluation. The result of the fit is a set of coefficients for the list of markers in the group. For example, A1AT (I1), A2M (I2), apolipoprotein CIII (I3), and TNF alpha (I4) were selected as the four markers representing the inflammatory group. Using binary logic regression against clinical results, four coefficients and the constants for these markers were calculated. The vector for the inflammatory group was constructed as follows:

V_{infla=}1(1+exp-(CI0+CI1*I1+Cl2*I2+CI3*I3+CI4*I4)) (6) (6)

Where CI0=-7.34
CI1=-0.929
Cl2=1.10
CI3=5.13
CI4=6.48

V_{infla} represented the probability of whether a given patient had MDD using the measured inflammatory markers.

In the same way, vectors for other groups of markers were derived for MDD. Four markers were chosen to represent the metabolic group: M1=ASP, M2=prolactin, M3=resistin, and M4=testosterone. Using the same method of binary logistic regression described above for the clinical data, a set of coefficients and a vector summary were developed for patient metabolic response:

Vₘₑₜₐ₌1/(1+exp-(Cm0+Cm1*M1+Cm2*M2+Cm3*M3+Cm4*M4)) (7)

Where Cm0=-1.10
Cm1=0.313
Cm2=2.66
Cm3=0.82
Cm4=-1.87

Vₘₑₜₐ represented the probability of whether a given patient had MDD using the measured metabolic markers.

Two markers were chosen to represent the HPA group: H1=EGF and H2=G-CSF. Again, using the same method of binary logistic regression on the clinical data as above, a set of coefficients and a vector summary were developed for patient HPA response:

Vₕₚₐ₌1/(1+exp-(Ch0+Ch1*H1+Ch2*H2)) (8)

Where Ch0=-1.87
Ch1=7.33
Ch2=0.53

Vₕₚₐ represented the probability of whether a given patient has MDD using the measured HPA markers.

Using these three parameters, a hypermap representation of patients diagnosed with MDD and a normal subject control group was constructed and shown in FIG. 5.

Certain external factors, disease or therapeutics, can influence the expression of one or more biomarkers that are components of a vector within a hypermap. FIG. 6 is a hypermap developed to demonstrate the response pattern for a series of MDD patients who initiated therapy with the antidepressant LEXAPRO™. FIG. 6 shows changes in BHYPERMAP™ in a subset of Korean MDD patients after treatment with LEXAPRO™. Data for MDD patients at baseline are represented by filled circles. Data points after two to three weeks of treatment are represented by filled triangles, and data points after eight weeks of treatment are represented by open squares. Open circles represent data for normal subjects. This demonstrates that the technology can be used to define changes in an individual pattern in response to antidepressant therapy.

### Example 5

### Diagnostic Markers of Depression

Methods as described herein were used to develop an algorithm for determining depression scores that are useful to, for example, diagnose MDD, stratify disease severity, and/or evaluate a patient's response to anti-depressive therapeutics. This systematic, highly parallel, combinatorial approach was proposed to assemble “disease specific signatures” using algorithms as described herein. Two statistical approaches were used for biomarker assessment and algorithm development: (1) univariate analysis of individual analyte levels, and (2) linear discriminant analysis and binary logistic regression for algorithm construction.

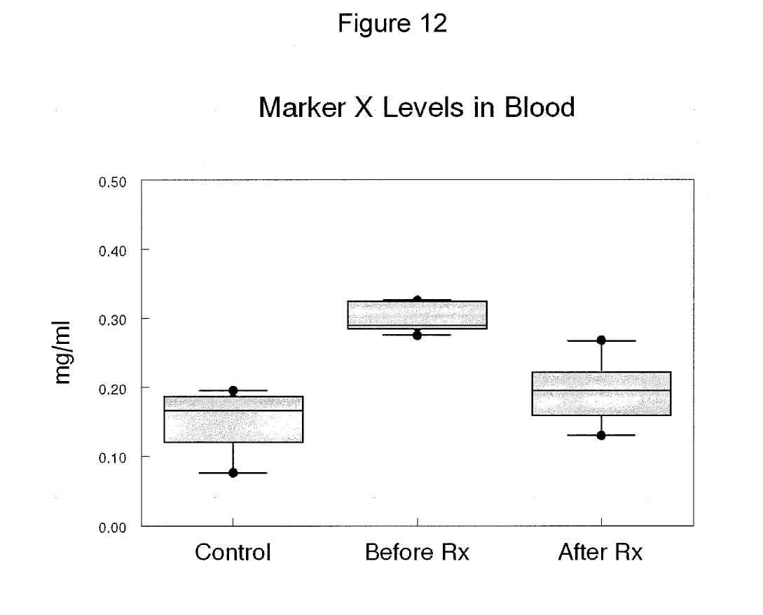

Univariate Analysis of Individual Analyte Levels: Univariate analysis explores each variable in a data set separately. This analysis looks at the range of values, as well as the central tendency of the values, describes the pattern of response to the variable, and describes each variable on its own. By way of example, FIG. 12 shows the distribution of blood levels of marker X in a hypothetical series of six MDD patients before and after treatment. The first point to be made from this graph is that the concentration of marker X was higher in untreated MDD patients as opposed to control subjects. Secondly, after treatment, the levels of marker X in the MDD patients were similar to that of the control. The Student's t-Test was then used to compare two sets of data and to test the hypothesis that a difference in their means was significant. Using the Student's t-Test, serum levels of each of the analytes tested using Luminex multiplex technology (xMAP) were analyzed for comparison of depressed versus normal subjects. Statistical significance was defined as p<0.05. Application of the Student's t-Test to the data, where there were equal numbers of points, showed that the difference in marker X expression between control subjects and patients with MDD was statistically significant (p=0.002), and the difference pre- and post-treatment also was statistically significant (p =0.013). There was no statistically significant difference between the control group and the MDD patients after treatment (p=0.35).

Algorithm Based On Linear Discriminant Analysis: In order to identify the analytes that contribute most to discrimination between classes (e.g., depressed versus normal subjects), a stepwise method of linear discriminant analysis (LDA) from SPSS Statistics v. 11.0 software (SPSS, Inc.) for the Microsoft WINDOWS® operating system was used with the following settings: Wilks' lambda (A) method was used to select analytes that maximize cluster separation and analyte entrance into the model was controlled by its F-value. A large F-value indicates that the level of the particular analyte is different between two groups, and a small F-value (F<1) indicates that there is no difference. In this method, the null hypothesis is rejected for small values of Λ. Thus, the aim was to minimize Λ.

To construct a list of analyte predictors, the F-values for each of the analytes was calculated. Starting with the analyte having the largest F-value (i.e., the analyte that differed most between the two groups), the value of Λ was determined. The analyte with the next largest F-value was then added to the list and Λ was recalculated. If the addition of the second analyte lowered the value of Λ, it was kept in the list of analyte predictors. The process of adding analytes one at a time was repeated until the reduction of Λ no longer occurred.

Cross-validation, a method for testing the robustness of a prediction model, was then carried out. To cross-validate a prediction model, one sample was removed and set aside. The remaining samples were used to build a prediction models based on the pre-selected analyte predictors. A determination was made as to whether the new model was able to predict the one sample not used in building the new model correctly. This process was repeated for all samples, one at a time, to calculate a cumulative cross-validation rate. The final list of analyte predictors was determined by manually entering and removing analytes to maximize the cross-validation rate, using information obtained from the univariate analyses and cross-validations. The final analyte classifier was then defined as the set of analyte predictors that gave the highest cross-validation rate.

Choosing Multiple Biomarkers for Major Depressive Disorder: Serum levels of approximately 100 analytes were tested using Luminex multiplex technology. The Student's t-test was used to compare data for depressed individuals versus normal subjects. Statistical significance was defined as α<0.05. After the initial study, statistically significant analytes were selected for further study. Table 5 lists the chosen group of biomarkers and indicates how each relates to pathways observed to be altered in subjects with unipolar depression. These analytes were subjected to multivariate analysis (PCA, PLS-DA, and LDA) to identify biomarkers that can be used to distinguish MDD patients from normal populations.

TABLE 5

| Gene Symbol | Gene Name | Pathway |
|---|---|---|
| A1AT | Alpha 1 Antitrypsin | Inflammation |
| A2M | Alpha 2 Macroglobin | Inflammation |
| ApoC3 | Apolipoprotein CIII | Inflammation |
| CD40L | CD40 ligand | Inflammation |
| IL-1 | Interleukin 1 | Inflammation |
| IL-6 | Interleukin 6 | Inflammation |
| IL-13 | Interleukin 13 | Inflammation |
| IL-18 | Interleukin 18 | Inflammation |
| IL-1ra | Interleukin 1 Receptor Antagonist | Inflammation |
| MPO | Myeloperoxidase | Inflammation |
| PAI-1 | Plasminogen activator inhibitor-1 | Inflammation |
| RANTES | RANTES (CCL5) | Inflammation |
| sTNFR2 | Soluble TNF-α Receptor II | Inflammation |
| TNFA | Tumor Necrosis Factor alpha | Inflammation |
| None | Cortisol | HPA axis |
| EGF | Epidermal Growth Factor | HPA axis |
| GCSF | Granulocyte Colony Stimulating Factor | HPA axis |
| PPY | Pancreatic Polypeptide | HPA axis |
| ACTH | Adrenocorticotropic hormone | HPA axis |
| AVP | Arginine Vasopressin | HPA axis |
| CRH | corticotropin-releasing hormone | HPA axis |
| ACRP30 | Adiponectin | Metabolic |
| ASP | Acylation Stimulating Protein | Metabolic |
| FABP | Fatty Acid Binding Protein | Metabolic |
| INS | Insulin | Metabolic |
| LEP | Leptin | Metabolic |
| PRL | Prolactin | Metabolic |
| RETN | Resistin | Metabolic |
| None | Testosterone | Metabolic |
| TSH | Thyroid Stimulating Hormone | Metabolic |
| BDNF | Brain-derived neurotrophic factor | \Neurotrophic |
| S100B | S100B | Neurotrophic |
| NTF3 | Neurotrophin 3 | Neurotrophic |
| GDNF | Glial cell line derived neurotrophic | Neurotrophic |
| | factor | |
| ARTN | Artemin | Neurotrophic |

Using nine of the markers listed in Table 5, a diagnostic score was established for each subject based on the following algorithm:

Depression diagnostic score (MDDSCORE™)=f(a1*Cortisol+a2*Prolactin+a3*EGF+a4*MPO+a5*BDNF+a6*Resistin+a7*sTNFR2+a8*ApoC3+a9*A1AT

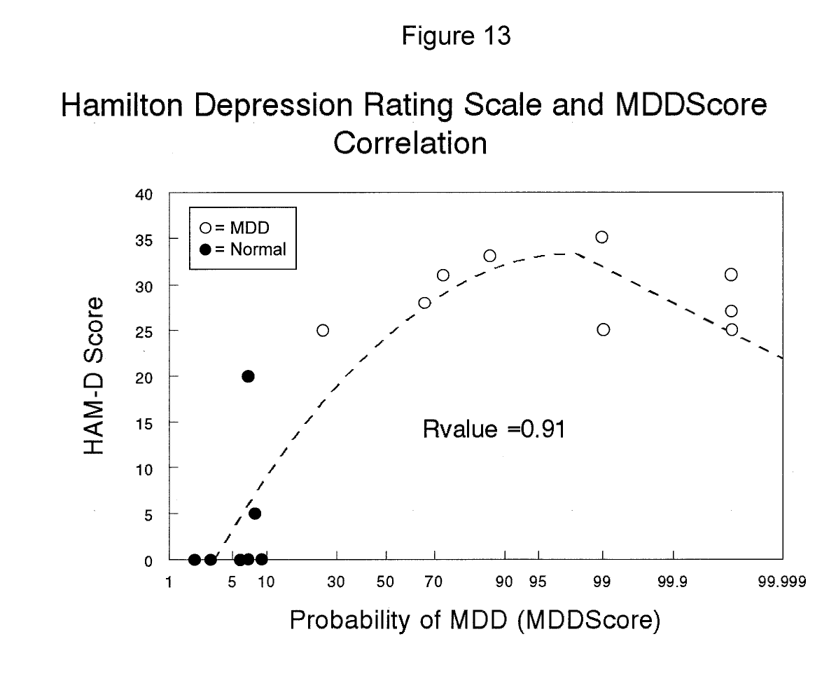

FIG. 13 shows the correlation between the depression diagnostic score and the HAM-D score. The HAM-D is a 21-question multiple choice questionnaire that clinicians can use to rate the severity of a patient's major depression. Support for the view that higher depression rating scale scores do predict a difference in outcome emerged from a review of the U.S. Food and Drug Administration database of 45 clinical trials of antidepressants. This study found that for both the investigational antidepressant [usually a selective serotonin reuptake inhibitor or serotonin-specific reuptake inhibitor (SSRI)] and the active comparator [usually a tricyclic antidepressant (TCA)], the trials with higher mean baseline HAM-D scores were associated with greater reductions in HAM-D scores at the end of a 4- to 8-week trial than trials with a lower mean baseline HAM-D.

### Example 6

### Depression Diagnostic Scores Change Following Drug Therapy

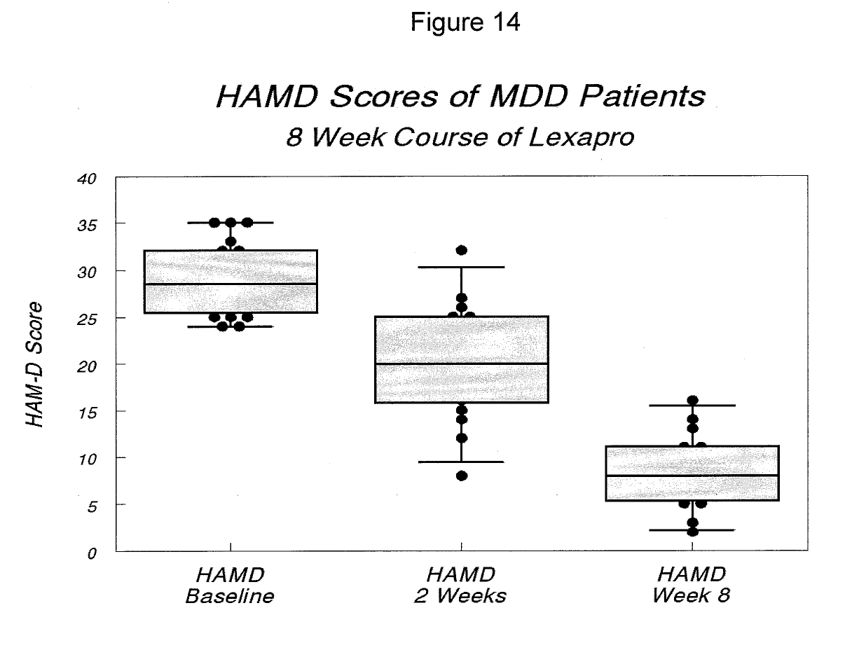

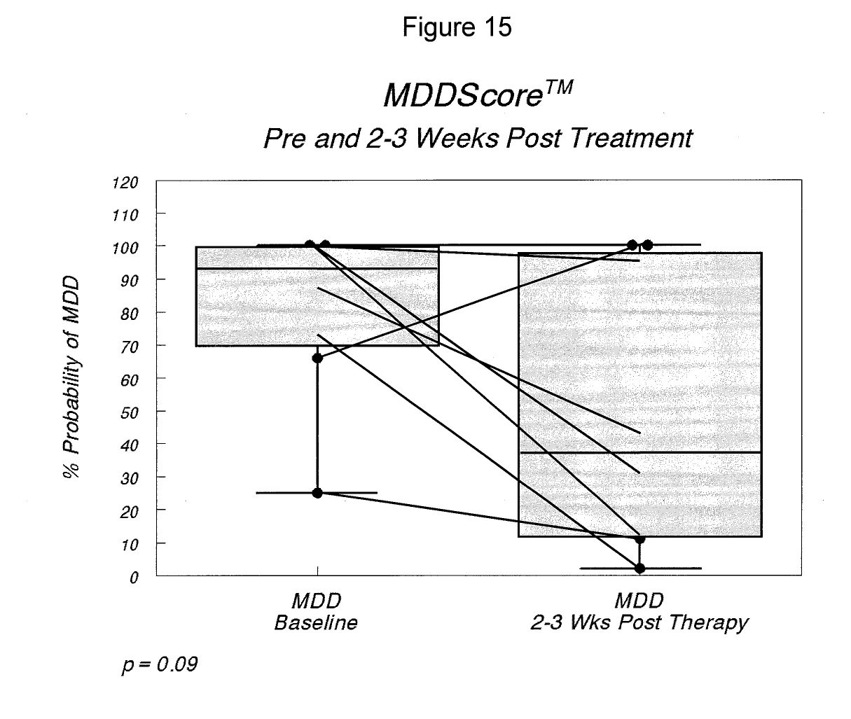

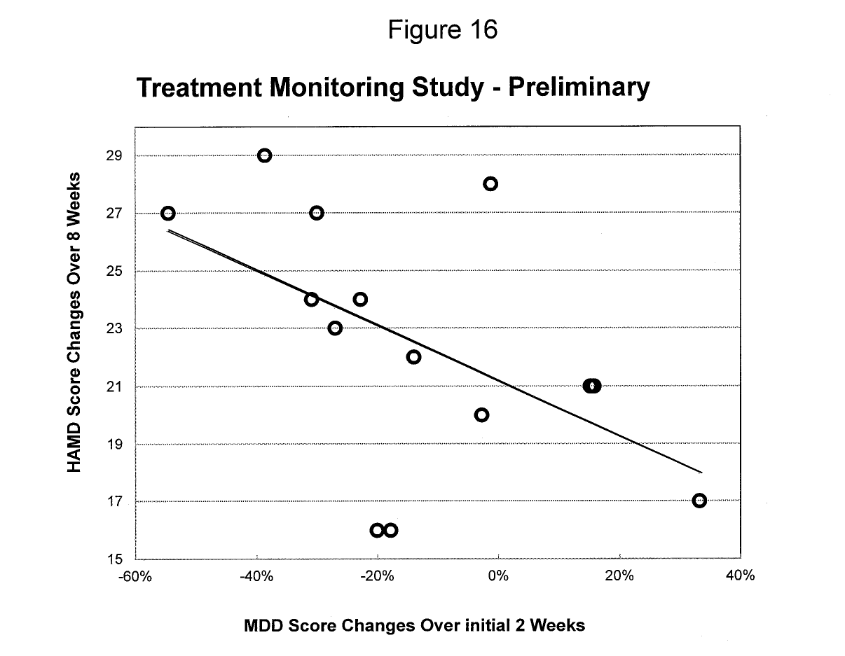

Using the algorithms described herein to establish diagnostic scores, patient populations were stratified according to HAM-D scores above 25. FIG. 14 indicates that patient HAM-D Scores improved (i.e., reduced) at both 2 and 8 weeks after treatment with the antidepressant Lexapro (a SSRI). FIG. 15 shows the change in MDDSCORE™ in a subset of those patients at baseline and after 2 weeks of treatment. FIG. 16 shows the potential for predicting the efficacy of treatment at 8 weeks by determining the MDDSCORE™ after 2 weeks of treatment. These data are indicative of treatment efficacy and demonstrate the utility of MDD diagnostic scores for both patient stratification and treatment monitoring.

### Example 7

### Antidepressant Treatment Monitoring with Multiple Biomarker Measurements

To develop an algorithm for using a panel of biomarker measurements to monitor antidepressant drug treatment, a group of patient candidates was selected for antidepressant drug treatment, and an initial blood sample was taken from each patient. The samples were spun down to separate serum from cells, and stored as PS1 (Patient p draw 1). Each patient was treated with an antidepressant drug (Lexapro®) for eight weeks, and blood samples were collected during the course of treatment. The samples were spun down, labeled and stored.

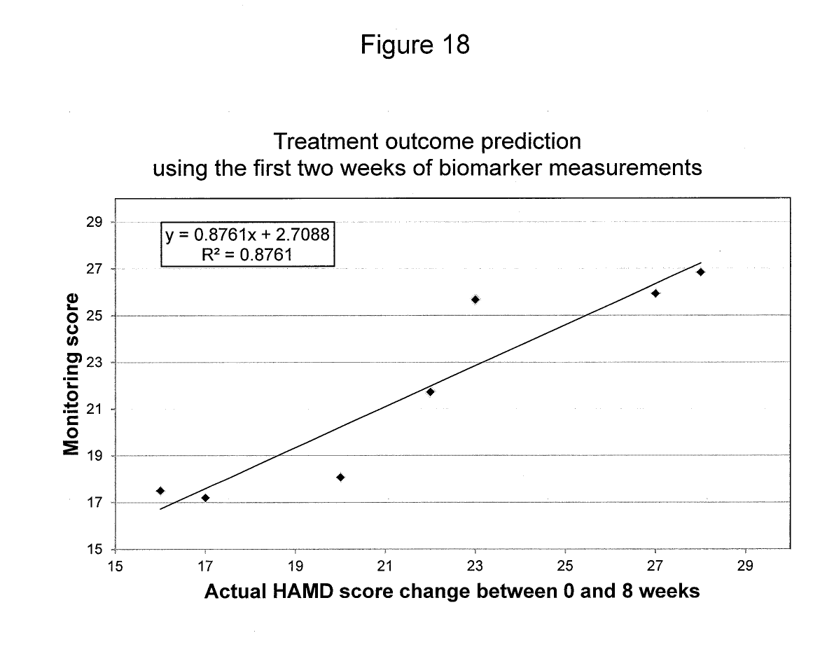

The samples (PS1, PS2, PS3, etc.) for each patient were assayed to measure the levels of five biomarkers—prolactin, BDNF, resistin, TNFRII, and A1A (Mn1, Mn2, and Mn3=biomarkers n1, n2, and n3; FIG. 17). A mathematical algorithm was applied to the biomarker measurements to calculate a monitoring score that was correlated to the final outcome (the HAMD score change) at the end of the antidepressant treatment period. The mathematical algorithm used the specific biomarker changes and the rates of those changes to calculate the score. In particular, the differences in biomarker measurements from two samples (an initial sample and a sample obtained two weeks into treatment) were measured, and a mathematical algorithm was used to calculate a monitoring score to predict the final treatment outcome (indicated by the HAMD score changes between an initial HAMD score and a HAMD score calculated after 8 weeks of treatment). See, FIG. 18.

At end of treatment, the outcome for each patient was known (i.e., whether treatment is successful). This result was used as an input to optimize the calculation that used biomarker measurements (Mn1, Mn2, Mn3, etc.) to predict patient treatment results. Comparing the clinical outcome with the biomarker measurements optimized generation of a monitoring score that maximally correlates to the treatment outcome for a patient treated with an antidepressant drug.

The marker panel included biomarkers selected from four major biological systems/pathways (inflammation, HPA axis, metabolic biomarkers, and neurotrophic factors, as described herein). Other exemplary biomarker panels that are used in the methods described herein include the following:
a biomarker panel including cortisol, PRL, BDNF, RES, TNFR, and A1A;
a biomarker panel including S100B, PRL, BDNF, RES, TNFR, and A1A;
a biomarker panel including RANTES, PRL, BDNF, S100B, RES, TNFR, and A1A;
a biomarker panel including RANTES, PRL, BDNF, S100B, RES, TNFR, AlA, and EGF; and
a biomarker panel including RANTES, PRL, BDNF, S100B, RES, TNFR, A1A, cortisol, and EGF.

### Example 8

### Identification of Pharmacodynamic Biomarkers Associated with MDD

FIG. 3 illustrates a process for identifying pharmacodynamic biomarkers of MDD. A collection of biomarkers that have a potential association with MDD was selected based on the result of earlier studies, from a literature search, from genomic or proteomic analysis of biological pathways, or from molecular imaging studies. A cohort of MDD patients was identified using a “gold standard” method of interview-based clinical assessment. Forty depressed adult subjects were enrolled at three Medical Centers in South Korea following IRB approval of the protocol. Enrolled subjects were 18 to 65 years old, met the DSM-IV criteria for Unipolar Major Depression, (single or recurrent), had a 17-item HAM-D score >16, and were capable of providing informed consent. All subjects were psychoactive drug-free for at least 6 months at study start and had the Structural Clinical Interview for DSM-IV (SCID) at baseline. Plasma or serum samples were collected from each patient, and patients were then subjected to treatment with escitalopram (e.g., LEXAPRO™, Forest Laboratories, New York, N.Y). Post-treatment plasma or serum samples were collected from each patient at two and eight weeks post-treatment. In addition, HAM-D and MADRS were assessed at baseline and after. De-identified plasma and serum samples were frozen at −80° C. before analysis.

Biomarker levels were tested using immunoassay methods. For example, serum or plasma levels of A1AT, ApoCIII, ASP, BDNF, cortisol, EGF, MPO, PRL, RES, S100B, and sTNFαRII in peripheral blood were measured using ELISAs according to manufacturer instructions. A1AT was measured using a human A1AT immunoassay (BioVendor, Candler, N.C.); ApoCIII was measured using a human ApoCIII immunoassay (AssayPro, St. Charles, Mo.); BDNF, sTNFαRII, and EGF levels were determined using Quantikine human ELISA kits from R&D Systems (Minneapolis, Minn.); MPO was measured using a human serum ELISA kit obtained from ALPCO Immunoassays (Salem, N.H.); PRL in serum was measured using a human serum ELISA from Monobind (Lake Forest, Calif.); and cortisol levels in serum were determined using a competition ELISA from IBL-America; Minneapolis, Minn.). S100B and ASP were laboratory developed tests (LDTs) developed at Ridge Diagnostics. Biomarker depression scores (MDDSCORE™, ranging from 1 to 9 and indicating low to high likelihood of depression) were determined (see, e.g., U.S. patent application Ser. No. 12/753,022, which is incorporated herein by reference in its entirety).

The panel was validated in a study of 123 subjects (80 depressed and 43 normal). The panel discriminated patients with MDD from normal controls (p=5.8e⁻¹⁹) and showed a clinical sensitivity of 87% and specificity of 95%. This panel and a second 6-biomarker panel, designed to include markers that were most likely to change with successful treatment, were further studied in a separate cohort of depressed patients to explore the ability of the panels to predict treatment outcomes.

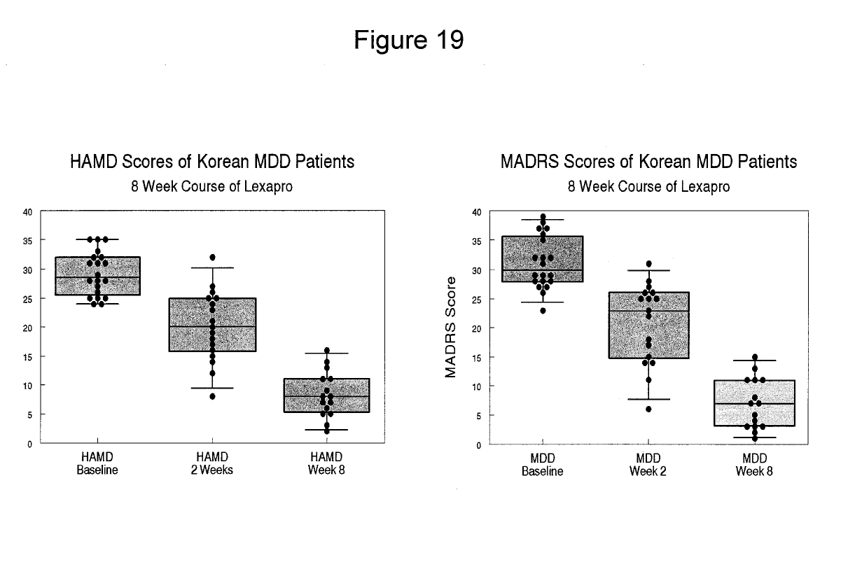

Patient response to treatment, determined by conducting additional structured clinical interviews and assigning post-treatment diagnostic scores, were recorded. Patients demonstrating a positive clinical response to treatment, which was defined as an improved (lower) post-treatment diagnostic score relative to the pre-treatment baseline score, were identified. Two clinical assessment tools (HAM-D and MADRS) were applied to the study population described above. Serum samples were obtained at baseline and at two and eight weeks post-treatment. As expected for a positive response to therapy, patients' scores on both tools decreased over the course of treatment (FIG. 19).

Analytes whose expression correlated with positive clinical outcomes were identified as pharmacodynamic biomarkers for MDD.

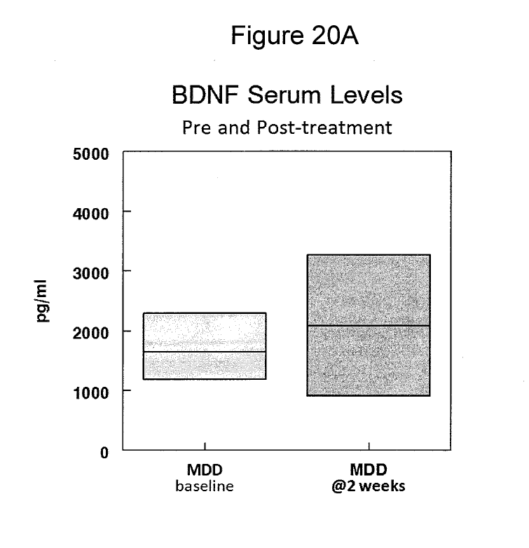

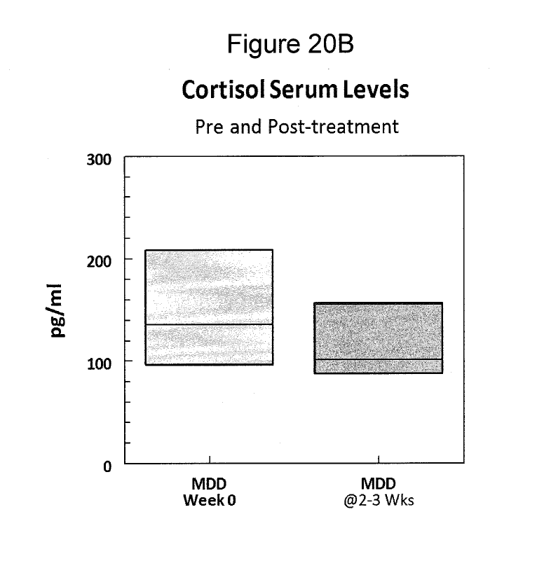

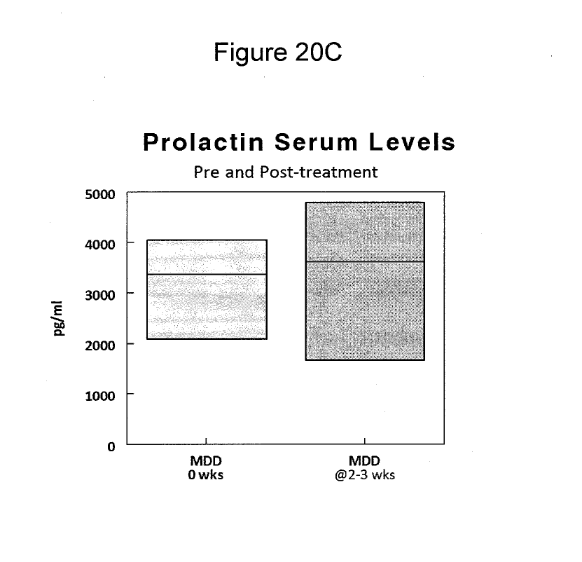

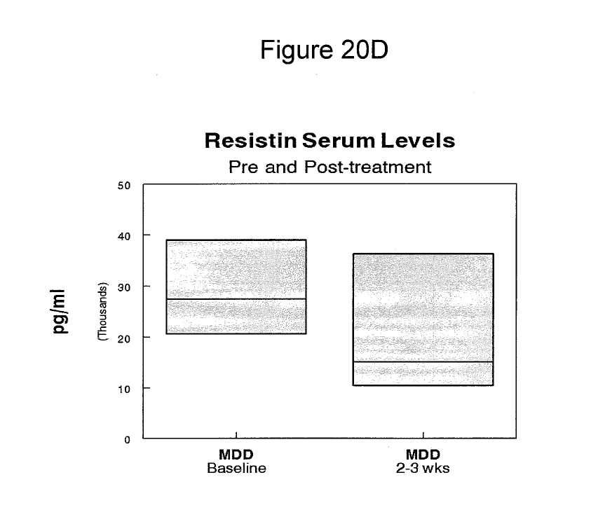

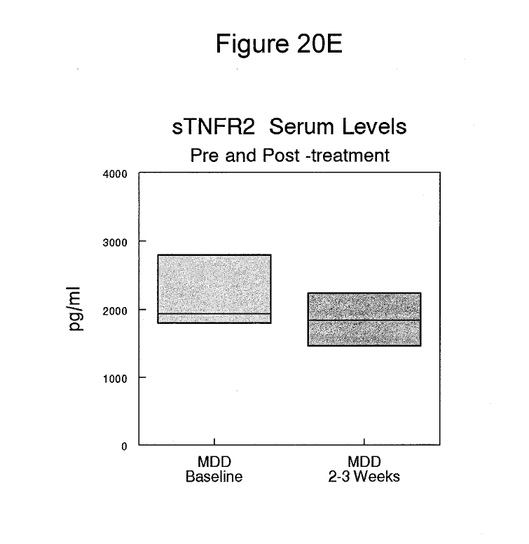

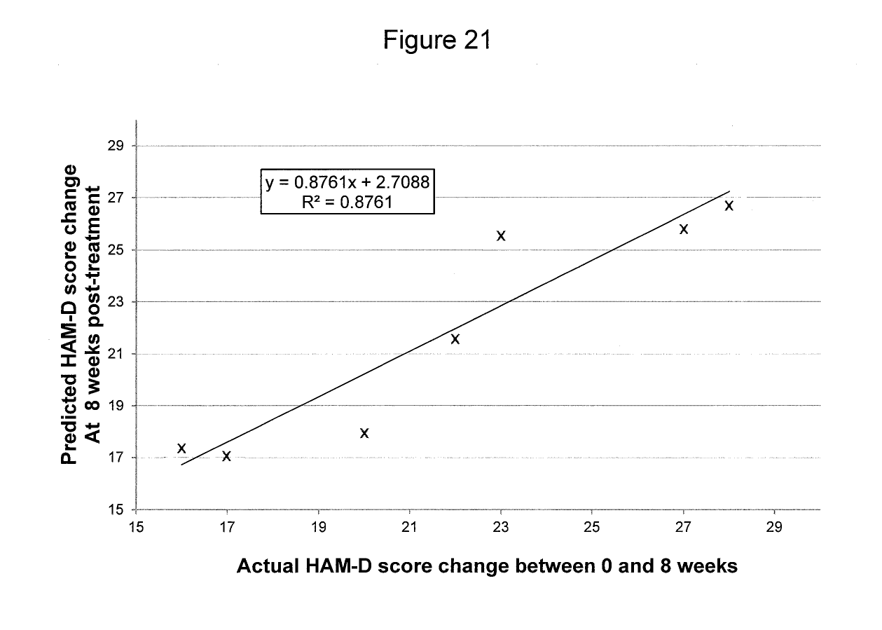

Following the assessment of 96 possible markers, a final “monitoring” panel of markers, including neurotrophic, metabolic, inflammatory, and HPA axis markers, was selected. The test consisted of A1AT, ApoC3, BDNF, cortisol, EGF, MPO, PRL, RES, and sTNFαRII. Levels of BDNF, cortisol, PRL, RES, and sTNFαRII are plotted in FIGS. 20A-20E, respectively. Results for a composite “monitoring panel” (PRL, BDNF, RES, sTNFαRII, and A1AT) at baseline and at week two were evaluated by regression analysis with the change in HAM-D score from baseline to week eight (FIG. 21). This analysis yielded a correlation coefficient of 0.88, suggesting that the monitoring biomarker panel values at week two may have the potential to predict therapy outcome at week eight.

This study in a small cohort of depressed patients suggests the utility of multi-analyte biomarker panels for the prediction of patient response to antidepressant therapy. This is a unique approach to the prediction of patient treatment outcome and it has the advantage of providing a serum-based, objective result that appears to correlate well with standard measures of patient treatment response to antidepressants. However, these findings are limited by the small sample size and larger studies in well-defined depressed patient populations will be needed to validate these early observations.

### Example 9

### Using Proteomics to Analyze Multiple Biomarkers

As shown in FIG. 3, treatment-relevant biomarkers are identified using tandem mass spectrometry. Biological samples are collected pre- and post-treatment. The samples are labeled with different Tandem Mass Tags (TMT) and mixed for TMT-MS™ (Proteome Sciences, United Kingdom). Following fragmentation/digestion with a suitable enzyme (e.g., trypsin), TMT labeled fragments are selected for analysis by liquid chromatography MS/MS. The ratio of protein expression between samples is revealed by MS/MS by comparing the intensities of the individual reporter group signals. Bioinformatic analysis is used to determine the proteins that are differentially expressed. The identified proteins are then validated as potential biomarkers (e.g., using specific antibodies, and ELISA) over a defined period of time after treatment to establish a subset of pharmacodynamic biomarkers. Statistical analysis of a subject's changes in analyte expression levels is performed to correlate analytes with treatment efficacy. Upon statistical evaluation where statistical significance is defined as p<0.05, biomarkers having a p value less than 0.05 are selected as biomarkers associated with therapy-responsive MDD.

### OTHER EMBODIMENTS

While this document contains many specifics, these should not be construed as limitations on the scope of an invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or a variation of a subcombination.

Only a few embodiments are disclosed. Variations and enhancements of the described embodiments and other embodiments can be made based on what is described and illustrated in this document.

1-40. (canceled)

41. A method for assessing a treatment response in a mammal having a neuropsychiatric disease, comprising:
(a) determining a first diagnostic disease score for said mammal, wherein said first diagnostic disease score is calculated using numerical values for the levels of at least two inflammatory markers, at least two HPA axis markers, and at least two metabolic markers present in a first biological sample obtained from said mammal prior to administration of said treatment;
(b) determining a second diagnostic disease score for said mammal, wherein said second diagnostic disease score is calculated using numerical values for the levels of at least two inflammatory markers, at least two HPA axis markers, and at least two metabolic markers present in a second biological sample obtained from said mammal after administration of said treatment; and
(c) maintaining, adjusting, or stopping said treatment of said mammal based on a comparison of said first diagnostic disease score to said second diagnostic disease score.

42. The method of claim 41, wherein said mammal is a human.

43. The method of claim 41, wherein said treatment is transcranial magnetic stimulation.

44. The method of claim 41, wherein said first diagnostic disease score is calculated using numerical values for the levels of at least two inflammatory markers, at least two HPA axis markers, at least two metabolic markers, and at least two neurotrophic markers present in said first biological sample.

45. The method of claim 41, wherein said second diagnostic disease score is calculated using numerical values for the levels of at least two inflammatory markers, at least two HPA axis markers, at least two metabolic markers, and at least two neurotrophic markers present in said second biological sample.

46. The method of claim 41, wherein said method comprises using a hypermap that comprises using a score for said levels of said inflammatory markers, a score for said levels of said at least two HPA axis markers, and a score for said levels of said at least two metabolic markers to compare said first and second diagnostic disease scores.

47-68. (canceled)

69. A method for monitoring treatment of a subject diagnosed with a depressive disorder, comprising:
(a) providing a first numerical value of each of two or more analytes selected from the group consisting of prolactin (PRL), brain derived neurotrophic factor (BDNF), resistin (RES), soluble tumor necrosis factor alpha receptor type II (sTNFαRII), alpha-1 antitrypsin (A1AT), apolipoprotein CIII (ApoC3), cortisol, epidermal growth factor (EGF), S100B, and myeloperoxidase (MPO), wherein each first numerical value corresponds to the level of the analyte in a first biological sample from the subject;
(b) individually weighting each first numerical value in a manner specific to each analyte to obtain a first weighted value for each analyte;
(c) determining a first MDD score based on an equation that includes each first weighted value;
(d) providing a second numerical value for each of the two or more analytes, wherein each second numerical value corresponds to the level of the analyte in a second biological sample from the subject, wherein the second biological sample is obtained after treatment for the depressive disorder;
(e) individually weighting each second numerical value in a manner specific to each analyte to obtain a second weighted value for each analyte, with the proviso that the weighting is done in a manner comparable to that in step (b);
(f) using the equation to determine a second MDD score after treatment of the subject for the depressive disorder; and
(g) comparing the first MDD score to the second MDD score and to a control MDD score or range of MDD scores determined from one or more normal subjects, and classifying the treatment as being effective if the second MDD score is closer than the first MDD score to the control MDD score, or classifying the treatment as not being effective if the second MDD score is not closer than the first MDD score to the control MDD score.

70. The method of claim 69, wherein step (a) comprises providing a first numerical value for three or more analytes selected from the group consisting of PRL, BDNF, RES, sTNFαRII, A1AT, ApoC3, cortisol, EGF, S100B, and MPO, and wherein step (d) comprises providing a second numerical value for each of the three or more analytes.

71. The method of claim 69, wherein step (a) comprises providing a first numerical value for four or more analytes selected from the group consisting of PRL, BDNF, RES, sTNFαRII, A1AT, ApoC3, cortisol, EGF, S100B, and MPO, and wherein step (d) comprises providing a second numerical value for each of the four or more analytes.

72. The method of claim 69, wherein step (a) comprises providing a first numerical value for five or more analytes selected from the group consisting of PRL, BDNF, RES, sTNαaRII, A1AT, ApoC3, cortisol, EGF, S100B, and MPO, and wherein step (d) comprises providing a second numerical value for each of the five or more analytes.

73. The method of claim 69, wherein the two or more analytes are PRL, BDNF, RES, sTNFαRII, and A1AT.

74. The method of claim 69, wherein the neuropsychiatric disease is major depressive disorder (MDD).

75. The method of claim 69, wherein the first and second biological samples are blood samples.

76. The method of claim 69, wherein the treatment is behavioral therapy.

77. The method of claim 69, wherein the treatment comprises drug therapy.

78. The method of claim 69, wherein the treatment comprises group therapy, interpersonal therapy, psychodynamic therapy, relaxation therapy, or traditional psychotherapy.

79. (canceled)
